SEQUENCE LISTING (114 sequences)

SEQ ID NO: 1 - protein, 255 aa, Nitrososphaera viennensis
MSQQRASFSGELVLGSPELAKILTYPRGSDKEYAARLAELKGLGVTAVFAGGRTVIGGTSIAGKGCVGLVVRAKAHGKMCALKIRRMDANRPTMHDEVRYHKIANGAGVGPRLVGYSDNFMLMEFAEGATIAEWAQGEIDGKQASAVTRSALEQCYALDRAGLDHGELSHVDRHIIVSGVQAATTIIDFESASTERKASNVSAAGQSLLVSGAVASALARVLQVEKEAAIGALKKYKRDQTRENLDAILALVVVA

SEQ ID NO: 2 - protein, 191 aa, Nautilia profundicola
MIIFEKKGTKIRYDIIEKIGEGNRGEVYKAKLEDGRVAAIKWAKNYEIDKEWEILSFLDGLCAPKPIYRGKRYFIMEYVDGKPLKEYIGSSEYYEVLKKALHNAYVLDEKGVFHGQLGRYYHILNTAREVKFIDFERGVFTQNPRNFLQILGYYLFRDEKYDKKALNLIVDLYKKNRKEALNKIIRLIDES

SEQ ID NO: 3 - protein, 255 aa, Leptolyngbya sp.
MAGSQFLTEDQENQLLGYPRISPVEQQARRQELTAFGLEGICQAGEQSLCDLQILGLGYVGLVVLVIREGKQFALKIRRTNAKRESLLPEAKAIRQANKVGVAPKIYQASDNFILMDFIEGKSFLEWLQWAIAKYPTSIILDVINNLLEQAYKLDQIGLDRDDMKCITKDVIVTATHQPVLLDFSNASGDRRPQNVTALVQGLFWGSVIAKYLKPLLPHCNQEQLLSHLRHYKGHPNRTNFDTLKQAITLSSLNL

SEQ ID NO: 4 - protein, 216 aa, Thermococcus kodakarensis
MTFEHIISSKRLEGFLRHLAEEGVGGVEPLAKGTTSLVFTGVLGGRKVVIKLQRPDSPRSNFEKEAELTKIASTFGVTPPIIGLGEFEGLPYLIREFAEGEPILFADVEKEHLFRIVEKTALLDRLGIDHGQIQGGKHIIIGEDVYLIDFEKAGFRKPNNLTSAMAMIFIGENAISKRVREKFGLDEKFREEMKDALRHYKRTGSLSRLLSLLSGL

SEQ ID NO: 5 - protein, 199 aa, Sulfurisphaera tokodaii
MRDFIYPRYDENIERELIEHGIKELYSFGPINLGKVNVIGKGKTGIVVLVDDNKVIKIRRSDSPKETLEIEAKIQIKAFPVAPKIHDYGRNFILMEYIDGRHLTREEKIDIIIDLLRKAKELEDKKIEHKELARPYKNVIVKPDRVYIIDYDSASFKENPLNVTSILSWLNFPHLANMYKKHRDIEEIVSLLYLLNKNQ

SEQ ID NO: 6 - protein, 253 aa, Nitrosopumilus maltimus
MAHSFISIKKFVDEPYSEILGYPKSTPRQTKSRINELDKLKIKSISLTGPTTLGKLEILGKGYVGVVVLAKKGSREVALKIRRIDSQRKEMKSEAKLLKLVNSVNVGPKMIDVSKNFLVMEYIEGIKIVDWVNSLKGVGSVKKLKSTIRKVLEDCYNLDQIGFDHGELSNISKHVIVGKTKSTLIDFESSSVKRRPSNVTSVTQAIFIGSGIAKKVQKIYKNPPKEKIIGALKQYKQEKSLENFENLLKILKL

SEQ ID NO: 7 - protein, 225 aa, Methanococcus vanielli
MTNLDFKMNIEGLNAISSKLFDWEILKNLSEYIVFTEYVGKGHRGVVFKAFSDKYIDKNGNHIILAVKIPRLDAPKVTIPNEGRILKKTNEFGVGPKVYEYSENHMVMEYVDGEMLKDCIDNLTPEELLYVIEETLRQCLRLDLHKIDHTEIQGGKHIMVSKKGVYIIDFDKAREHSPKNFTSAMSLLFGENYISKKIMHLLNLSEEKIILFRKYAKNYKTLFKN

SEQ ID NO: 8 - protein, 192 aa, Aquifex aeolicus
MKFSEFIKEVQNLQELAEGWRGKVYTGYWRGKKVAIKVAKAPDKVKAIQKEAEILEKLKGLKGFPQILFKGEDFFVYEFIEGKPFGKLKIGEEEKKRLLKEVLEKAYLLDRMGINRDEFSNIYKNVLVGDKGEVFVIDFDRGTFKKNPSNVRQFLQLLKREGFLSQEEAIELGKRYKENPEEVIKEIRKVLS

SEQ ID NO: 9 - RNA, 10 nt, Escherichia coli
  source 1..10: mol_type = tRNA
aggtctgcgg

SEQ ID NO: 10 - RNA, 10 nt, Escherichia coli
  source 1..10: mol_type = tRNA
aggtcagcgg

SEQ ID NO: 11 - RNA, 10 nt, Escherichia coli
  source 1..10: mol_type = tRNA
cggtcggagg

SEQ ID NO: 12 - RNA, 10 nt, Escherichia coli
  source 1..10: mol_type = tRNA
gggtcgcggg

SEQ ID NO: 13 - RNA, 10 nt, Escherichia coli
  source 1..10: mol_type = tRNA
gggtcgcgag

SEQ ID NO: 14 - RNA, 10 nt, Escherichia coli
  source 1..10: mol_type = tRNA
ttgtcgtggg

SEQ ID NO: 15 - RNA, 10 nt, Escherichia coli
  source 1..10: mol_type = tRNA
aggtcggtgg

SEQ ID NO: 16 - RNA, 10 nt, Escherichia coli
  source 1..10: mol_type = tRNA
tggtcgctgg

SEQ ID NO: 17 - RNA, 10 nt, Escherichia coli
  source 1..10: mol_type = tRNA
tggtcgcagg

SEQ ID NO: 18 - RNA, 10 nt, Escherichia coli
  source 1..10: mol_type = tRNA
aggtcgtcgg

SEQ ID NO: 19 - RNA, 10 nt, Escherichia coli
  source 1..10: mol_type = tRNA
gggtcacagg

SEQ ID NO: 20 - RNA, 10 nt, Escherichia coli
  source 1..10: mol_type = tRNA
gggtcggagg

SEQ ID NO: 21 - RNA, 10 nt, Escherichia coli
  source 1..10: mol_type = tRNA
aggtcgtagg

SEQ ID NO: 22 - RNA, 10 nt, Escherichia coli
  source 1..10: mol_type = tRNA
aggtcggcag

SEQ ID NO: 23 - RNA, 10 nt, Escherichia coli
  source 1..10: mol_type = tRNA
gggtcggcgg

SEQ ID NO: 24 - RNA, 10 nt, Escherichia coli
  source 1..10: mol_type = tRNA
gggtcggtgg

SEQ ID NO: 25 - RNA, 10 nt, Escherichia coli
  source 1..10: mol_type = tRNA
gggtcgttgg

SEQ ID NO: 26 - DNA, 35 bp, synthetic construct
  source 1..35: mol_type = other DNA
agggcggcgtcctaaccaggctagacgacgggccc

SEQ ID NO: 27 - DNA, 40 bp, synthetic construct
  source 1..40: mol_type = other DNA
gttaaaagcccgccgctctacctggctgagctacgggccc

SEQ ID NO: 28 - DNA, 40 bp, synthetic construct
  source 1..40: mol_type = other DNA
ttacaaggccggcgctctaaccaggctgagctacagcggc

SEQ ID NO: 29 - DNA, 35 bp, synthetic construct
  source 1..35: mol_type = other DNA
ggccagcgtcctaatccagactagacgacggccgc

SEQ ID NO: 30 - DNA, 40 bp, synthetic construct
  source 1..40: mol_type = other DNA
tccgaaggccggcgctctatcctggctgagctacgggtcc

SEQ ID NO: 31 - DNA, 35 bp, synthetic construct
  source 1..35: mol_type = other DNA
ggcctgcatcctagtccaggctagacgacggtccc

SEQ ID NO: 32 - DNA, 36 bp, synthetic construct
  source 1..36: mol_type = other DNA
tgcggcccgccgccttaacctctcggccaccccggc

SEQ ID NO: 33 - DNA, 40 bp, synthetic construct
  source 1..40: mol_type = other DNA
ggccaaagccctcgatccttgaccactagacgacccggct

SEQ ID NO: 34 - DNA, 40 bp, synthetic construct
  source 1..40: mol_type = other DNA
cctaagcccgacccctttgaccttgctcgggcacccccgc

SEQ ID NO: 35 - DNA, 40 bp, synthetic construct
  source 1..40: mol_type = other DNA
ctctcaaggccagccccttagtccactcgggcaccccggc

SEQ ID NO: 36 - DNA, 35 bp, synthetic construct
  source 1..35: mol_type = other DNA
gagccgggcgctctaccgggctaagctacgggccc

SEQ ID NO: 37 - DNA, 35 bp, synthetic construct
  source 1..35: mol_type = other DNA
gtcttcagccgggcgctctcccgggctgagctacg

SEQ ID NO: 38 - DNA, 31 bp, synthetic construct
  source 1..31: mol_type = other DNA
gactgactcatatgcgccaggtactacaaaa

SEQ ID NO: 39 - DNA, 33 bp, synthetic construct
  source 1..33: mol_type = other DNA
gactctcgagtatcttttcgagcggtatgtttc

SEQ ID NO: 40 - DNA, 37 bp, synthetic construct
  source 1..37: mol_type = other DNA
aaacttgtacttccaaggaatgaccttcgagcatatc

SEQ ID NO: 41 - DNA, 39 bp, synthetic construct
  source 1..39: mol_type = other DNA
attcggatcccatatgggatcacagaccgctcagtaaag

SEQ ID NO: 42 - DNA, 41 bp, synthetic construct
  source 1..41: mol_type = other DNA
aaacttgtacttccaaggaatggccattaaaaaagaaattc

SEQ ID NO: 43 - DNA, 43 bp, synthetic construct
  source 1..43: mol_type = other DNA
attcggatcccatatgggatcacagttttttataggttttggc

SEQ ID NO: 44 - DNA, 32 bp, synthetic construct
  source 1..32: mol_type = other DNA
gtgatctgggatccatgtctcaacagagagcc

SEQ ID NO: 45 - DNA, 34 bp, synthetic construct
  source 1..34: mol_type = other DNA
gtgatctggcggccgctcaggcaacaacaacaag

SEQ ID NO: 46 - DNA, 41 bp, synthetic construct
  source 1..41: mol_type = other DNA
aaacttgtacttccaaggaatgaaatttagcgagttcatta

SEQ ID NO: 47 - DNA, 37 bp, synthetic construct
  source 1..37: mol_type = other DNA
attcggatcccatatgggatcagctcagcactttgcg

SEQ ID NO: 48 - DNA, 42 bp, synthetic construct
  source 1..42: mol_type = other DNA
ccgcgaacagattggaggtatgatcatcttcgagaaaaaggg

SEQ ID NO: 49 - DNA, 44 bp, synthetic construct
  source 1..44: mol_type = other DNA
cttggaagtacaagttttctcagctttcatcaatcaggcgaata

SEQ ID NO: 50 - DNA, 38 bp, synthetic construct
  source 1..38: mol_type = other DNA
ccgcgaacagattggaggtatggcaggtagccagtttc

SEQ ID NO: 51 - DNA, 40 bp, synthetic construct
  source 1..40: mol_type = other DNA
cttggaagtacaagttttctcacagattcaggctgctcag

SEQ ID NO: 52 - DNA, 37 bp, synthetic construct
  source 1..37: mol_type = other DNA
aaacttgtacttccaaggaatgaagccagagcggaag

SEQ ID NO: 53 - DNA, 40 bp, synthetic construct
  source 1..40: mol_type = other DNA
attcggatcccatatgggactaaaccgccagggttataca

SEQ ID NO: 54 - DNA, 16 bp, synthetic construct
  source 1..16: mol_type = other DNA
tcccatatgggatccg

SEQ ID NO: 55 - DNA, 19 bp, synthetic construct
  source 1..19: mol_type = other DNA
tccttggaagtacaagttt

SEQ ID NO: 56 - DNA, 22 bp, synthetic construct
  source 1..22: mol_type = other DNA
gaaaacttgtacttccaaggat

SEQ ID NO: 57 - DNA, 18 bp, synthetic construct
  source 1..18: mol_type = other DNA
acctccaatctgttcgcg

SEQ ID NO: 58 - DNA, 34 bp, synthetic construct
  source 1..34: mol_type = other DNA
gcttgcatgcctgcagtccttccataggtagatg

SEQ ID NO: 59 - DNA, 39 bp, synthetic construct
  source 1..39: mol_type = other DNA
actatttaagcttatcgcgtcaagagaaccggaagcctc

SEQ ID NO: 60 - DNA, 20 bp, synthetic construct
  source 1..20: mol_type = other DNA
acgcgataagcttaaatagt

SEQ ID NO: 61 - DNA, 35 bp, synthetic construct
  source 1..35: mol_type = other DNA
cctctagagtcgaccagtccaaaccctcgatataa

SEQ ID NO: 62 - DNA, 37 bp, synthetic construct
  source 1..37: mol_type = other DNA
ccaagcttgcatgcctgcaaccatccgcaaaggggag

SEQ ID NO: 63 - DNA, 37 bp, synthetic construct
  source 1..37: mol_type = other DNA
gaaactcgcatgggctgacgctaaaaacctcacggga

SEQ ID NO: 64 - DNA, 19 bp, synthetic construct
  source 1..19: mol_type = other DNA
gtcagcccatgcgagtttc

SEQ ID NO: 65 - DNA, 38 bp, synthetic construct
  source 1..38: mol_type = other DNA
ggatcctctagagtcgaccctttgctatcaaaagccgc

SEQ ID NO: 66 - DNA, 20 bp, synthetic construct
  source 1..20: mol_type = other DNA
ggtcgactctagaggatccc

SEQ ID NO: 67 - DNA, 18 bp, synthetic construct
  source 1..18: mol_type = other DNA
tgcaggcatgcaagcttg

SEQ ID NO: 68 - DNA, 41 bp, synthetic construct
  source 1..41: mol_type = other DNA
cagtaatacgactcactatagggcccgtggtctagatggtt

SEQ ID NO: 69 - DNA, 48 bp, synthetic construct
  source 1..48: mol_type = other DNA
ccgtggtctagatggttatgacgccacccttacaaggtggaggtccgg

SEQ ID NO: 70 - DNA, 41 bp, synthetic construct
  source 1..41: mol_type = other DNA
tggtgggcccgcggggattcgaaccccggacctccaccttg

SEQ ID NO: 71 - DNA, 38 bp, synthetic construct
  source 1..38: mol_type = other DNA
cagtaatacgactcactatagggcgcgtggtctagatg

SEQ ID NO: 72 - DNA, 57 bp, synthetic construct
  source 1..57: mol_type = other DNA
agggcgcgtggtctagatggttatgacgccacccttacaaggtggaggtccggggtt

SEQ ID NO: 73 - DNA, 40 bp, synthetic construct
  source 1..40: mol_type = other DNA
tggtgggcgcgcggggattcgaaccccggacctccacctt

SEQ ID NO: 74 - DNA, 28 bp, synthetic construct
  source 1..28: mol_type = other DNA
gctggcagcgggtaccacctctttagtg

SEQ ID NO: 75 - DNA, 28 bp, synthetic construct
  source 1..28: mol_type = other DNA
gtacccgctgccagcggttccacaccac

SEQ ID NO: 76 - DNA, 29 bp, synthetic construct
  source 1..29: mol_type = other DNA
gcaaaggcgaccacctctttagtgtttac

SEQ ID NO: 77 - DNA, 27 bp, synthetic construct
  source 1..27: mol_type = other DNA
ggtggtcgcctttgccagcggttccac

SEQ ID NO: 78 - DNA, 28 bp, synthetic construct
  source 1..28: mol_type = other DNA
ggtgattgcgctgcagcgcccggacagc

SEQ ID NO: 79 - DNA, 28 bp, synthetic construct
  source 1..28: mol_type = other DNA
gctgcagcgcaatcaccactttgcgacc

SEQ ID NO: 80 - DNA, 27 bp, synthetic construct
  source 1..27: mol_type = other DNA
gaaaaagcagctgagctgaccaaaatc

SEQ ID NO: 81 - DNA, 27 bp, synthetic construct
  source 1..27: mol_type = other DNA
ctcagctgctttttcaaaattgctacg

SEQ ID NO: 82 - DNA, 28 bp, synthetic construct
  source 1..28: mol_type = other DNA
ctgattgcggaatttgccgagggcgaac

SEQ ID NO: 83 - DNA, 28 bp, synthetic construct
  source 1..28: mol_type = other DNA
caaattccgcaatcagatacggtaaacc

SEQ ID NO: 84 - DNA, 27 bp, synthetic construct
  source 1..27: mol_type = other DNA
attgatgctggccagatccaaggtggc

SEQ ID NO: 85 - DNA, 27 bp, synthetic construct
  source 1..27: mol_type = other DNA
ctggccagcatcaatgcccagacgatc

SEQ ID NO: 86 - DNA, 27 bp, synthetic construct
  source 1..27: mol_type = other DNA
catggcgcgatccaaggtggcaaacac

SEQ ID NO: 87 - DNA, 27 bp, synthetic construct
  source 1..27: mol_type = other DNA
ttggatcgcgccatgatcaatgcccag

SEQ ID NO: 88 - DNA, 27 bp, synthetic construct
  source 1..27: mol_type = other DNA
ggtggcgcgcacattatcatcggcgag

SEQ ID NO: 89 - DNA, 27 bp, synthetic construct
  source 1..27: mol_type = other DNA
aatgtgcgcgccaccttggatctggcc

SEQ ID NO: 90 - DNA, 27 bp, synthetic construct
  source 1..27: mol_type = other DNA
ctgatcgcgtttgagaaggccggcttc

SEQ ID NO: 91 - DNA, 27 bp, synthetic construct
  source 1..27: mol_type = other DNA
ctcaaacgcgatcagatacacatcctc

SEQ ID NO: 92 - DNA, 27 bp, synthetic construct
  source 1..27: mol_type = other DNA
ccgaacgcgctgaccagcgccatggcc

SEQ ID NO: 93 - DNA, 27 bp, synthetic construct
  source 1..27: mol_type = other DNA
ggtcagcgcgttcggtttgcggaagcc

SEQ ID NO: 94 - DNA, 28 bp, synthetic construct
  source 1..28: mol_type = other DNA
aatctggcgagcgccatggccatgatct

SEQ ID NO: 95 - DNA, 27 bp, synthetic construct
  source 1..27: mol_type = other DNA
ggcgctcgccagattgttcggtttgcg

SEQ ID NO: 96 - DNA, 27 bp, synthetic construct
  source 1..27: mol_type = other DNA
cgccacgcgaaacgcaccggtagtctg

SEQ ID NO: 97 - DNA, 28 bp, synthetic construct
  source 1..28: mol_type = other DNA
gcgtttcgcgtggcgtaaagcatctttc

SEQ ID NO: 98 - DNA, 27 bp, synthetic construct
  source 1..27: mol_type = other DNA
cactacgcgcgcaccggtagtctgagt

SEQ ID NO: 99 - DNA, 27 bp, synthetic construct
  source 1..27: mol_type = other DNA
ggtgcgcgcgtagtggcgtaaagcatc

SEQ ID NO: 100 - DNA, 29 bp, synthetic construct
  source 1..29: mol_type = other DNA
actacaaagcgaccggtagtctgagtcgt

SEQ ID NO: 101 - DNA, 27 bp, synthetic construct
  source 1..27: mol_type = other DNA
accggtcgctttgtagtggcgtaaagc

SEQ ID NO: 102 - DNA, 44 bp, synthetic construct
  source 1..44: mol_type = other DNA
cagtaatacgactcactatagggcggctggtctagctcggttat

SEQ ID NO: 103 - DNA, 44 bp, synthetic construct
  source 1..44: mol_type = other DNA
ctggtctagctcggttatgataccgctcttacacagcggtggtc

SEQ ID NO: 104 - DNA, 41 bp, synthetic construct
  source 1..41: mol_type = other DNA
tgggcggcgggtgatttgaacacccgaccaccgctgtgtaa

SEQ ID NO: 105 - DNA, 41 bp, synthetic construct
  source 1..41: mol_type = other DNA
ctggtctagctcggttatgataccgctcttacacagcggtg

SEQ ID NO: 106 - DNA, 44 bp, synthetic construct
  source 1..44: mol_type = other DNA
tgggcggcgggtgatttgaacacccggccaccgctgtgtaagag

SEQ ID NO: 107 - DNA, 44 bp, synthetic construct
  source 1..44: mol_type = other DNA
tgggcggcgggtgatttgaacacccgtccaccgctgtgtaagag

SEQ ID NO: 108 - DNA, 44 bp, synthetic construct
  source 1..44: mol_type = other DNA
tgggcggcgggtgatttgaacacccgcccaccgctgtgtaagag

SEQ ID NO: 109 - DNA, 37 bp, synthetic construct
  source 1..37: mol_type = other DNA
gctaatacgactcactataagccgggtagtctagtgg

SEQ ID NO: 110 - DNA, 62 bp, synthetic construct
  source 1..62: mol_type = other DNA
agccgggtagtctagtggtcaaggatccagggctttggcccctgggaccagggttcgaatcc

SEQ ID NO: 111 - DNA, 32 bp, synthetic construct
  source 1..32: mol_type = other DNA
tggtagccgggcagggattcgaaccctggtcc

SEQ ID NO: 112 - protein, 184 aa, Escherichia coli
MAKYNEKELADTSKFLSFVLRHKPEAIGIVLDREGWADIDKLILCAQKAGKRLTRALLDTVVATSDKKRFSYSSDGRCIRAVQGHSTSQVAISFAEKTPPQFLYHGTASRFLDEIKKQGLIAGERHYVHLSADEATARKVGARHGSPVILTVKAQEMAKRGLPFWQAENGVWLTSTVAVEFLEW

SEQ ID NO: 113 - protein, 180 aa, Thermococcus kodakarensis
MKPERKRVSKLMAYILRHSPEEFGLRPDVEGFVSLNELVNALKTVYPEVTEEFVREIVENDPKGRYEIRGDRIRARYGHSFPVSLDHEEDTESRFLYHGTPRRNLPSILKEGLKPMKRQYVHVSTDKIEALETGRRHGREVVLLVIDAECLRKRGFKIYKAGKNVRIVERVPPDCITLAV

SEQ ID NO: 114 - protein, 230 aa, Saccharomyces cerevisiae
MRQVLQKDKRDVQLSKALSYLLRHTAVKEKLTIDSNGYTPLKELLSHNRLKTHKCTVDDIHRIVKENDKQRFHIKTLGADEEWICATQGHSIKSIQPSDEVLVPITEASQLPQELIHGTNLQSVIKIIESGAISPMSRNHVHLSPGMLHAKGVISGMRSSSNVYIFIDCHSPLFFQTLKMFRSLNNVYLSSSIPVELIQKVVVKGNLKDEEKLDTLRRILHERNIPLEKI

(19) United States
(12) Patent Application Publication
(10) Pub. No.: US 2025/0304929 A1
(43) Pub. Date: Oct. 2, 2025

(54) Enzyme Phosphorylating 2' Hydroxyl Group of RNA

(71) Applicant: The University of Tokyo, Tokyo (JP)

(72) Inventors: Tsutomu Suzuki, Tokyo (JP); Takayuki Ohira, Tokyo (JP); Keiichi Minowa, Tokyo (JP)

(21) Appl. No.: 18/853,539

(22) PCT Filed: Apr. 10, 2023
(86) PCT No.: PCT/JP2023/014605

§ 371 Date: Oct. 2, 2024

Related U.S. Application Data
(60) Provisional application No. 63/328,775, filed on Apr. 8, 2022.

(51) Int. Cl. C12N 9/12; C12P 19/34

(52) U.S. Cl. CPC C12N 9/12; C12P 19/34; C12Y 207/11

(57) ABSTRACT

The present invention provides an enzyme and the like that phosphorylates a 2′ hydroxyl group of RNA.

### CROSS-REFERENCE TO RELATED APPLICATIONS

This application claims the benefit of U.S. Provisional Patent Application No. 63/328,775, filed with the United States Patent and Trademark Office on Apr. 8, 2022, the content of which is incorporated herein by reference in its entirety.

The entire content of Ohira et al., “Reversible RNA phosphorylation stabilizes tRNA for cellular thermotolerance” (Nature volume 605, pages 372-379 (2022)) is incorporated herein by reference.

### TECHNICAL FIELD

Protein synthesis (translation) is an essential process for all organisms. During the translation process, tRNA functions as an adaptor molecule that associates the genetic code (codon) on mRNA with amino acids. tRNA undergoes various chemical modifications after transcription and becomes functionally mature. These modifications contribute to the stabilization of the three-dimensional structure of tRNA and the accuracy of codon decoding. Therefore, it is known that defects or abnormalities in tRNA modifications lead to breakdown of gene expression and often cause poor growth and diseases. To date, about 150 types of RNA modifications have been found in various species (Non-Patent Document 1).

In addition, an example is known in which tRNA modifications in thermophilic organisms that grow in a high-temperature environment are involved in heat resistance of tRNA, and thermophilic organisms lacking RNA modifications exhibit high temperature sensitivity. tRNA modifications of which modification rate changes dynamically depending on the growth temperature are also known, and regulating of the rigidity and flexibility of tRNA contributes to optimizing translation at the growth temperature.

### BACKGROUND ART

### Citation List

### Non-Patent Document

Non-Patent Document 1: Nucleic Acids Research, Volume 46, Issue D1, 4 Jan. 2018, Pages D303-D307

### SUMMARY

### Technical Problem

Dynamic regulation of functions and metabolisms of proteins and various metabolites in living organisms by phosphorylation and dephosphorylation is a well-known basic concept in biochemistry and molecular biology, but the phosphorylation modification of RNA has been overlooked until now.

An object of the present invention is to provide a novel enzyme or the like that introduces a phosphorylation modification into RNA.

### Solution to Problem

The inventors found 2′ phosphorylated uridine (U^{p}) (U^{p} modification) that is a novel reversible RNA modification in tRNA, which is an adaptor molecule for protein synthesis, at position 47 in a variable loop of tRNA derived from hyperthermophilic archaea.

The inventors additionally identified a novel enzyme ArkI as an enzyme (writer) that introduces a U^{p} modification.

That is, the present application encompasses the following inventions.

[1] An enzyme phosphorylating a 2′ hydroxyl group of RNA.

[2] The enzyme according to [1], which is derived from archaea or bacteria.

[3] The enzyme according to [1] or [2], which is an RNA kinase.

[4] The enzyme according to any one of [1] to [3],
wherein the enzyme is any one of proteins (i) to (iii) below:
(i) a protein consisting of any of amino acid sequences SEQ ID NOs. 1 to 8;
(ii) a protein consisting of an amino acid sequence having 80% or more identity to any of amino acid sequences SEQ ID NOs. 1 to 8 and having an activity of phosphorylating the 2′ hydroxyl group of RNA; and
(iii) a protein consisting of an amino acid sequence in which one or several amino acids are deleted, substituted, or added in any of amino acid sequences SEQ ID NOs. 1 to 8 and having an activity of phosphorylating the 2′ hydroxyl group of RNA.

[5] The enzyme according to [4], wherein phosphorylation activity is ATP-dependent or GTP-dependent, and the 2′ hydroxyl group is at position 2′ of uridine.

[6] A nucleic acid stabilizer comprising the enzyme according to any one of [1] to [5] as an active ingredient.

[7] The stabilizer according to [6], wherein the nucleic acid is tRNA having uridine, adenosine, guanosine or cytidine at position 47 or RNA that is able to form a tRNA-like structure.

[8] The stabilizer according to [7], wherein the stabilization is stabilization against heat or resistance to RNase.

[9] A vector comprising a nucleic acid encoding the enzyme according to any one of [1] to [5].

[10] The vector according to [9], further including a nucleic acid encoding MTO1 (mitochondrial tRNA translation optimization 1).

[11] A kit comprising:
1) the enzyme according to any one of [1] to [5];
2) the stabilizer according to any one of [6] to [8]; or
3) the vector according to [9] or [10].

[12] The kit according to [11], further comprising uridine or a nucleic acid containing uridine, and optionally ATP.

[13] The kit according to or [12], further comprising
an enzyme that dephosphorylates the 2′ phosphate group of RNA, or optionally 2′ phosphorylated uridine or a nucleic acid containing 2′ phosphorylated uridine, and nicotinamide adenine dinucleotide (NAD⁺).

[154] The kit according to [13],
wherein the enzyme that dephosphorylates the 2′ phosphate group of RNA is a member of the KptA/Tpt1 family or a homologue thereof.

[15] The kit according to [11] to [14], further comprising tRNA.

[16] A composition comprising:
1) the enzyme according to any one of [1] to [5];
2) the stabilizer according to any one of [6] to [8]; or
3) the vector according to [9] or [10], and tRNA.

[17] A pharmaceutical composition comprising:
1) the enzyme according to any one of [1] to [5];
2) the stabilizer according to any one of [6] to [8]; or
3) the vector according to [9] or [10], and
an active ingredient for treating or preventing a tRNA-related disease.

[18] The pharmaceutical composition according to [17],
wherein the tRNA-related disease is a disease caused by reduced or deficient 5-taurinomethyluridine (τm⁵U) modification in mitochondrial tRNA.

[19] The pharmaceutical composition according to [18],
wherein the disease caused by reduced or deficient τm⁵U modification is mitochondrial encephalomyopathy (MELAS).

[20] A method for modifying a 2′ hydroxyl group of RNA, the method comprising
a step of bringing RNA into contact with the enzyme according to any one of [1] to [5].

[21] The method according to [20], further comprising
a step of additionally bringing RNA into contact with an enzyme that dephosphorylates 2′ phosphorylated uridine.

[22] The method according to or [21],
wherein the modification is phosphorylation or dephosphorylation.

[23] A method for stabilizing a nucleic acid containing uridine, the method comprising
a step of bringing a nucleic acid containing uridine into contact with the enzyme according to any one of [1] to [5].

[24] The method according to [23],
wherein thermostability or resistance to RNase is imparted to the nucleic acid.

[25] A method for producing 2′ phosphorylated uridine or a nucleic acid containing 2′ phosphorylated uridine, the method comprising a step of bringing uridine or a nucleic acid containing uridine into contact with the enzyme according to any one of [1] to [5].

### Advantageous Effects of Invention

X-ray crystal structure analysis of tRNA suggests a new function of RNA modification that the U^{p} modification prevents thermal denaturation of tRNA by allowing a metastable three-dimensional structure of tRNA. For example, the U^{p} modification imparts heat resistance and resistance to RNase to tRNA and thus contributes to heat resistance of organisms.

In order to clarify the mechanism by which the U^{p} modification stabilizes the tRNA structure, in collaboration with Professor Kozo Tomita (Department of Computational Biology and Medical Sciences, Graduate School of Frontier Sciences, The University of Tokyo), the inventors conducted X-ray crystal structure analysis and determined the three-dimensional structure of archaeal tRNA at a high resolution of 1.9 Å. It has been found that the U^{p} modification imparts the flexibility to the variable loop and also prevents rotation of the main chain and thus acts like a “padlock” to prevent the core region of tRNA from being thermally denatured. Actually, tRNA molecules having a non-standard core structure stabilized by the U^{p} modification have been found in the crystal structure. That is, a principle of stabilizing the RNA structure in which the U^{p} modification prevents thermal denaturation of tRNA by stabilizing a metastable core region structure, which was previously unknown, has been found.

Biochemical analysis showed that ArkI is a novel RNA kinase belonging to the protein kinase family, and kinetic analysis suggested that the rate-limiting factor for the phosphorylation reaction is the ATP concentration, and the U^{p} modification could be introduced by sensing the intracellular energy state. In addition, according to X-ray crystal structure analysis, the three-dimensional structure (1.8 Å) of ArkI was disassembled, and the structure of the active center and amino acid residues important for RNA recognition were identified.

The inventors identified KptA as a U^{p} modification dephosphorylation enzyme (eraser), and found through kinetic analysis that KptA has an activity of efficiently dephosphorylating the U^{p} modification from tRNA. In addition, the inventors found that KptA also acts as an eraser of the U^{p} modification in cells. This result strongly suggests that, in species having both ArkI and KptA, the structure and function of tRNA could be reversibly regulated by the U^{p} modification.

Dynamic regulation of functions and metabolisms of proteins and various metabolites in living organisms by phosphorylation and dephosphorylation is a well-known basic concept in biochemistry and molecular biology, but the phosphorylation modification of RNA has been overlooked until now. In this study, the reversible phosphorylation modification of RNA was found for the first time in the world, and it was clearly found that this modification regulated the function and structure of RNA and additionally contributed to environmental adaptation of extremophilic organisms. The result of this study contributes to understanding of a regulation mechanism of epitranscriptomic gene expression by RNA modifications and adaptive evolution of life through RNA modifications.

RNA has been receiving focus as a new modality in pharmaceutical development. Particularly, nucleic acid drugs such as mRNA vaccines, siRNA and aptamers are being put into practice. RNA modifications are known to have important functions such as stabilizing RNA drugs in cells and evading natural immunity, and the U^{p} modification reported in this study is expected to be applied to RNA drugs in the future. Particularly, when tRNA is modified by phosphorylation, since the heat resistance in protein synthesis is greatly improved, the phosphorylation modification of RNA is expected to be applied to RNA drugs and the like.

### BRIEF DESCRIPTION OF DRAWINGS

FIG. 1 shows diagrams in which the U^{p} modification imparts thermostability and resistance to RNase to tRNA. FIG. 1(A) shows the secondary structure of S. tokodaii tRNA^{Val3}, with position 47: U^{p} modification; position 15: archaeosine (G⁺) modification. FIG. 1(B) shows the chemical structure of 2′phosphorylated uridine (U^{p}) modification. FIG. 1(C) shows the thermal melting curve of S. tokodaii tRNA^{Val3}. Comparing tRNA with the U^{p} modification (U^{p}47) with tRNA (U47) without the U^{p} modification. The Tₘ value of tRNA without the U^{p} modification was lowered by 6.6° C. FIG. 1(D) shows sensitivity of S. tokodaii tRNA^{Val3} to RNase. tRNA (U47) without the U^{p} modification rapidly degraded over time, but tRNA with the U^{p} modification (U^{p}47) exhibited clear resistance to RNase.

FIG. 2 shows diagrams in which the U^{p} modification stabilizes a metastable core structure of tRNA. FIG. 2(A) shows the X-ray crystal structure of S. tokodaii tRNA^{Val3} (a resolution of 1.9 A). FIG. 2(B) shows structural comparison of the core region. Untreated S. tokodaii tRNA^{Val3} (U^{p}47), tRNA (U47) with the U^{p} modification removed, and yeast tRNA^{Phe} (U47, PDB: 1EHZ) were superimposed. The position 47 is shown as a Ball and Stick. In the untreated tRNA, the 2′ phosphate group faces the side of the solvent, and the uracil base faces the core region. FIG. 2(C) shows a diagram in which the U^{p} modification acts like a padlock to restrict the range of rotation of the main chain while providing a certain degree of flexibility to tRNA and to prevent tRNA from collapsing. FIG. 2(D) shows the model for stabilizing the tRNA core region by the U^{p} modification. Since thermal denaturation of tRNA starts when the core region collapses, the removal of G46 from the base triple composed of ψ13-G22-G46 can be considered as an intermediate state of thermal denaturation. It is thought that U^{p} modification not only contributes to stabilizing the core structure by restricting rotation of the main chain but also receives G46 dissociated from the core through stacking, and stabilizes a metastable core structure composed of ψ13-G22-C9, prevents tRNA from collapsing due to thermal denaturation, and increases the chance of restoring it to a standard core structure.

FIG. 3 shows diagrams in which the U^{p} modification contributes to high temperature adaptation of hyperthermophilic archaea. FIG. 3(A) shows a diagram in which the U^{p} modification is confirmed by LC/MS analysis. The top shows a chromatogram of UV absorbance (254 nm). The bottom shows a mass chromatogram of a fragment containing U^{p}47 (pU^{p}m⁵C, m/z 724). The left shows a wild type strain and the right shows a candidate gene knockout strain. The U^{p} modification disappeared in the candidate gene (ΔarkI) knockout strain. FIG. 3(B) shows the growth curve of the T. kodakarensis gene knockout strain (the culture temperature was 83° C., 87° C., and 91° C.). Wild type strain, arkI gene knockout strain (ΔarkI), the queE gene knockout strain involved in G⁺ modification (ΔqueE), and arkI/queE double gene knockout strain (ΔarkI/ΔqueE). arkI exhibited weak temperature sensitivity alone, and the double gene knockout strain exhibited significant temperature sensitivity. FIG. 3(C) shows a model of how the U^{p} modification stabilizes tRNA in cooperation with G⁺ modification. As described above, the U^{p} modification acts like a padlock that prevents tRNA from collapsing while allowing flexibility, whereas the G⁺ modification forms many interactions with the surrounding nucleotides, and stabilizes tRNA as tightly as a screw.

FIG. 4 shows diagrams in which ArkI is an ATP-dependent RNA kinase. FIG. 4(A) shows in vitro U^{p} modification reconstitution using recombinant TkArkI. The top shows a mass chromatogram of an RNA fragment containing U^{p}47. The bottom shows a mass chromatogram of an RNA fragment containing unmodified U47. The left shows the results when no ATP was added, and the right shows the results when ATP was added. FIG. 4(B) shows reaction kinetics analysis of U^{p} modification formation using TkArkI. The left shows the results in which the initial reaction rate with respect to the tRNA concentration was measured, and the Kₘ value and Vₘₐₓ value for tRNA were calculated by nonlinear analysis. The right shows the results in which the initial reaction rate with respect to the ATP concentration was measured, and the Kₘ value and Vₘₐₓ value for ATP were calculated in the same manner. FIG. 4(C) shows the X-ray crystal structure of TkArkI (a resolution of 1.8 A). A guanosine was bound to the active center (shown as a Ball and Stick). FIG. 4(D) shows the surface charge of TkArkI. A positively charged band is visible from the active center to the C-terminal lobe. FIG. 4(E) shows mutant analysis of TkArkI. The top shows the motif structure of TkArkI and the site into which a mutation was introduced. The bottom shows the activity of each mutant. The vertical axis represents the relative activity when the average activity of the wild type (WT) is set to 1. The horizontal axis represents the mutant in which each amino acid was substituted with alanine. Three trials were performed for each mutant, the average value is shown as a bar graph, the standard deviation is shown as an error bar, and the value for each trial is shown as a point. A large decrease in activity was observed in 11 mutants, which indicates that these amino acids contribute to activity.

FIG. 5 shows diagrams in which KptA is a U^{p} modification dephosphorylation enzyme. FIG. 5(A) shows the in vitro U^{p} modification dephosphorylation reaction using recombinant TkKptA. The top shows a mass chromatogram of an RNA fragment containing U^{p}47, and the bottom shows a mass chromatogram of an RNA fragment containing unmodified U47. The left shows the results when no TkKptA was added, and the right shows the results when TkKptA was added. FIG. 5(B) shows reaction kinetics analysis of the U^{p} dephosphorylation reaction using TkKptA. The initial reaction rate with respect to the tRNA concentration was measured, and the Kₘ value and Vₘₐₓ value for tRNA were calculated by nonlinear analysis. FIG. 5(C) shows the results in which, when TkArkI was expressed in E. coli and the U^{p} modification was introduced into endogenous tRNA in E. coli, expression of TkKptA was induced with different IPTG concentrations (0, 10, 100 UM). The top shows a mass chromatogram of an RNA fragment containing U^{p}47 (U^{p}CAGp), and the bottom shows a mass chromatogram of an RNA fragment (m⁵UψCGp) used as a control. FIG. 5(D) shows the results in which expression of TkKptA was induced with different IPTG concentrations, an RNA fragment containing a U^{p} modification (U^{p}CAGp) was then quantified, and the relative value with respect to the control RNA fragment is shown in a bar graph. The U^{p} modification was reduced according to expression of TkKptA, which indicates that KptA actually functions as an eraser in cells.

FIG. 6 shows the visualization result of ³²P-labeled tRNA. From the left, U at position 47: WT N. viennensis tRNA^{Val3}, C, A, G: N. viennensis tRNA^{Val3} in which position 47 was substituted with C, A, or G, and A: S. solfataricus tRNA^{Gln} lacking position 47. (Top) the SYBR Gold staining result. (Bottom) the results after 1 hour exposure to an imaging plate.

### DESCRIPTION OF EMBODIMENTS

### (Enzyme)

In a first embodiment, there is provided an enzyme that phosphorylates the 2′ hydroxyl group of RNA.

Nucleic acids that make up RNA and the like generally contain a pentose (ribose, deoxyribose, etc.), a base, and at least one phosphate group. There are two types of pentoses: D-ribose and deoxy-D-ribose, and ribonucleic acid (RNA) contains D-ribose, and deoxyribonucleic acid (DNA) contains deoxy-D-ribose. A compound in which a base is bound to position 1′ of D-ribose or deoxy-D-ribose is called a nucleoside, and a compound in which a phosphate is bound to position 5′ of a nucleoside via an ester bond is called a nucleotide. As used herein, “the 2′ hydroxyl group of RNA” is a hydroxyl group (2′ hydroxyl group) located at position 2 of a pentose (D-ribose) of a ribonucleotide.

The nucleotides may include deoxyribonucleotides, modified deoxyribonucleotides, ribonucleotides, modified ribonucleotides, peptide nucleotides, modified peptide nucleotides, modified phosphate-sugar framework nucleotides and mixtures thereof.

Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine monophosphate (dCMP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

Examples of ribonucleotides include ATP, UTP, CTP, GTP, ADP, UDP, CDP, GDP, AMP, UMP, CMP, and GMP, and examples of deoxyribonucleotides include dATP, dTTP, dCTP, dGTP, dADP, dTDP, dCDP, dGDP, dAMP, dTMP, dCMP, and GMP, and the present invention is not limited thereto.

The enzyme that phosphorylates the 2′ hydroxyl group of RNA is a protein that mediates the reaction of substituting the 2′ hydroxyl group with a phosphate group. Such a phosphorylation enzyme is also called a kinase. The kinase transfers a phosphate group from a phosphate group donor to a substrate.

Enzymes that phosphorylate the 2′ hydroxyl group of RNA can be classified as the AQ578 family, which is a protein kinase family (Leonard, C. J., Aravind, L. & Koonin, E. V. Novel families of putative protein kinases in bacteria and archaea: evolution of the “eukaryotic” protein kinase superfamily. Genome Res 8, 1038-1047 (1998).). Although the AQ578 family was predicted to be a serine/threonine kinase based on its sequence, it has been found to be an RNA kinase.

The base may be adenine (A), guanine (G), thymine (T), cytosine (C), or uracil (U). The base is not limited thereto and includes any natural or artificial nucleic acid base. Specific examples of bases other than the above bases include modified purine bases, for example hypoxanthine, xanthine, uric acid, 7-methylguanine, 2,6-diaminopurine, 6,8-diaminopurine, N6-methyladenine, 7-deazaxanthine, 7-deazaguanine; and modified pyrimidine bases, for example, 5,6-dihydrouracil, 5-methylcytosine, 5-hydroxymethylcytosine, N4, N4-ethanocytosine, 5-fluorouracil, and 5-bromouracil. The RNA contains at least one uridine as a base.

The ribonucleotides that make up RNA include adenosine 5′-phosphate (AMP), uridine 5′-phosphate (UMP), cytidine 5′-phosphate (CMP), and guanosine 5′-phosphate (GMP).

Various chemical modifications are enzymatically introduced into RNA after transcription. RNA modifications are universally located in various non-coding RNAs, including mRNA, tRNA, and rRNA, and these modifications provide important qualitative information that cannot be overlooked for RNA to function. RNA modifications change the three-dimensional positional relationship of bases and riboses, provide localized hydrophobic or hydrophilic sites, and may also contribute to formation and stabilization of higher-order structures of RNA. RNA modifications are also involved in interactions with RNA-binding proteins, controlling of RNA maturation and degradation, decoding of genetic codes, changing of genetic information, regulating of translation, and the like. RNA modifications also serve as marks for determining intracellular localization of RNA. In addition, RNA modifications are physiologically important because defects in RNA modifications cause severe diseases in humans.

About 150 types of RNA modifications have been found in various species up to now. About 80% of RNA modifications have been found in tRNA. The anticodon and its neighboring tRNA modification affect the codon decoding accuracy and protein synthesis efficiency. On the other hand, tRNA modifications found in the core region contribute to stabilizing the overall structure of tRNA, and improving heat resistance. Examples of groups added in RNA modifications include a methyl group, an acetyl group, an amino acid, a sugar, and taurine, and in this specification, an RNA modification such as addition of a phosphate group is called a phosphorylation modification.

It is known that eukaryotic mRNAs and non-coding RNAs have a cap structure at the 5′ end, and in recent years, various modifications such as inosine (I), 5-methylcytidine (m⁵C), N6-methyladenosine (m⁶A), 1-methyladenosine (m¹A), and pseudouridine (ψ) have been found through comprehensive analysis of transcriptomes using next-generation sequencing, and a new concept called epitranscriptome is being proposed. The epitranscriptome is a concept in which the structure and function of RNA are dynamically controlled by post-transcriptional modification of RNA, and is involved in various biological phenomena through the regulation of gene expression.

The RNA may be subjected to known chemical or biological modifications. Chemically modified forms include those in which a chemical moiety is bound to a nucleotide. Biologically modified forms may include those that have been post-transcriptionally modified, and those in which an amino acid is added to the 3′ end according to expression using prokaryotic host cells.

Enzymes that phosphorylate the hydroxyl groups of RNA are classified as RNA modification enzymes that introduce RNA modifications. RNA modification enzymes are also collectively called writers, with the nuance of writing RNA modifications. On the other hand, demethylases (RNA demodification enzymes) that erase modifications once written into RNA are called erasers, with the nuance of erasing modifications written by writers. Proteins that recognize RNA modifications are also called readers.

The RNA targeted by the enzyme that phosphorylates the 2′ hydroxyl group of RNA may be a coding RNA, for example, a messenger RNA (mRNA), and the RNA may be a non-coding RNA. Examples of non-coding RNAs include transfer RNA (TRNA), ribosomal RNA (rRNA), micro RNA (miRNA), small interfering RNA (siRNA), PIWI-interacting RNA (piRNA), small nucleolar RiboNucleoProtein (snoRNP), small nuclear RNA (snRNA), and extracellular RNA (ExRNA).

The RNA targeted by the enzyme that phosphorylates the 2′ hydroxyl group of RNA is preferably tRNA. tRNA is an RNA that acts as an adaptor molecule which matches codons (genetic code) with amino acids in protein synthesis. tRNA is a short single-stranded RNA with a length of 70 to 90 bases, and its secondary structure has a cloverleaf structure composed of three stem loops (D arm, anticodon arm, T arm) and one stem (acceptor stem). The loops that the D arm, anticodon arm, and T arm have are called a D loop, an anticodon loop, and a T loop (TYPC loop), respectively. Some tRNAs may contain an additional variable loop (V loop). The variable loop is located between the anticodon arm and the T arm. The structure of the center moiety of tRNA, which is formed of the D arm, T arm and variable loop, is called a core region. The length of each arm and the size of the loop of a tRNA molecule vary depending on species.

In one embodiment, the V loop has a length of at least 5 bases.

The anticodon arm has a region composed of three consecutive nucleotides called an anticodon, and the anticodon forms a base pair with a codon on mRNA to recognize the codon. On the other hand, a nucleic acid sequence (CCA sequence) composed of cytidine-cytidine-adenosine is present at the 3′ end of tRNA, and an amino acid is added to the adenosine residue at the end. The tRNA to which an amino acid is added is called aminoacyl-tRNA. In this specification, aminoacyl-tRNA is also included in tRNA.

tRNA has an L-shaped three-dimensional structure in which the above structure is folded. tRNA accepts the corresponding amino acid at the 3′ end, and there are different tRNA species corresponding to 20 types of amino acids. tRNA has an anticodon that pairs with a codon, the anticodon binds to a codon on mRNA (messenger RNA) with ribosomes, and thus the corresponding amino acid is introduced into the elongating protein.

Respective nucleosides in tRNA are numbered according to the tRNA numbering rule known in the art. For example, a typical tRNA is composed of 76 bases, but because the number of bases at a certain position increases depending on animal species, the anticodon is always numbered from positions 34 to 36. The variable region always starts at position 44, and has a length of 4 to 23 bases. The CCA sequence is always at positions 74 to 76.

Modified bases may be present at several positions throughout tRNA. The first anticodon base, which is at the “wobble-position,” may be modified into inosine (derived from adenosine), queuosine (derived from guanosine), uridine-5-oxyacetic acid (derived from uridine), 5-methylaminomethyl-2-thiouridine (derived from uridine), or lysidine (derived from cytidine).

In one embodiment, the phosphorylation modification is a modification in which uridine (U) (at position 2′) located at position 47 in the variable loop of tRNA is phosphorylated (2′ phosphorylated uridine: U^{p}).

The enzyme that phosphorylates the 2′ hydroxyl group of RNA may target tRNA having uridine, adenosine, guanosine or cytidine at position 47 or RNA that can form a tRNA-like structure. Such a tRNA is preferably a tRNA with a variable loop of several bases, for example, 2 to 9 bases, and at least 5 bases. In this specification, 2′ phosphorylated uridine at position 47 is also called “U^{p}47.”

Examples of RNA that can form a tRNA-like structure include mitochondrial tRNAs lacking the D arm or T arm; tRNA precursors having introns or immature end sequences; tRNA precursors formed by assembly of RNA fragments; RNA having a tRNA-like structure formed by assembly of different RNA molecules; tmRNA in bacteria; RNA having a tRNA-like structure found at the 3′ end of eukaryotic MALAT1 RNA; and RNA having a tRNA-like structure found in viral genomes, and the present invention is not limited thereto. RNA that can form a tRNA-like structure will be exemplified below in detail.

Among mitochondrial tRNAs, those having a small D loop in the secondary structure and those having no D arm or T arm are known. Although these tRNAs lack partial sequences, they have a tertiary structure similar to an L-shaped structure characteristic of tRNA, and function as tRNA. Therefore, these tRNAs can also be considered as “RNA that can form a tRNA-like structure.”

In some tRNA precursors, an intron sequence is inserted into the anticodon. The three-dimensional structure of such precursors is different from that of the mature form in that the length and direction of the anticodon arm change due to the intron, but the rest of the structure is the same as that of the mature form. Actually, ArkI can introduce a U^{p} modification into a precursor having an intron. Such a structure can also be considered as “RNA that can form a tRNA-like structure.”

As tRNA that archaea have, split-type tRNAs whose precursors are split into two or three RNAs are known, and these RNA fragments are assembled within cells and processed by cutting and linking to form a functional mature form. In addition, different tRNAs can be produced according to combinations of precursors. Such a tRNA precursor in a maturation procedure can also be considered as “RNA that can form a tRNA-like structure.”

It is known that short RNA fragments (for example, RNA fragments derived from Y4-RNA in human cells) are assembled with a target sequence within cells to form a tRNA-precursor-like structure, which is then processed with tRNA maturation enzymes. The RNA structure formed by interactions between such different types of RNA can also be considered as “RNA that can form a tRNA-like structure.”

RNA having a tRNA-like structure has been identified in various RNA molecules such as mRNA, non-coding RNA, and viral RNA in various species of bacteria and eukaryotes. These structures behave like tRNA and allow the reaction with enzymes that act on tRNA, and thereby contribute to expression of RNA functions and regulation of gene expression. Prokaryotic tmRNA is a chimeric RNA with properties of both tRNA and mRNA, which has a domain that forms a structure similar to tRNA and an mRNA-like domain, and has a function of helping ribosomes that have stopped synthesizing proteins. One having such a function as tRNA can also be considered as “RNA that can form a tRNA-like structure.”

The 3′ end side of eukaryotic MALAT1 RNA has a tRNA-like structure, and this structure is recognized by tRNA maturation enzymes and processed, and as maturation of MALAT1 RNA proceeds, the tRNA moiety is excised to become mascRNA. One having such a function as tRNA can also be considered as “RNA that can form a tRNA-like structure.”

It is known that the 3′ end side of turnip yellow mosaic virus (TYMV) has a tRNA-like structure, and aminoacylation of this end is related to the infectivity of this virus. One having such a function as tRNA can also be considered as “RNA that can form a tRNA-like structure.”

The enzymes that phosphorylate the 2′ hydroxyl group of RNA include not only those that occur naturally but also those that are artificially modified. The enzyme that phosphorylates the 2′ hydroxyl group of RNA may be derived from any of prokaryotes, eukaryotes, and archaea, including bacteria, but is often found in archaea that grow at a high temperature, for example, hyperthermophilic archaea that grow at a high temperature of 80° C. or higher or their related species. However, the origin of the enzyme that phosphorylates the 2′ hydroxyl group of RNA may be bacteria, for example, hyperthermophilic or moderately thermophilic bacteria or mesophilic bacteria. The species from which the enzyme that phosphorylates the 2′ hydroxyl group of RNA is derived is preferably a species that has a U^{p} modification.

Hyperthermophilic archaea are thought to be close to protocells because they are located at the base of the phylogenetic tree. Examples of hyperthermophilic archaea include archaea such as those belonging to the phylum Euryarchaeota, for example, the genus Thermococcus, the genus Pyrococcus, the genus Methanothermus, the genus Methanothermobacter, and the genus Methanocaldcoccus, and those belonging to the phylum Crenarchaeota, for example, archaea the genus Sulfurisphaera, the genus Sulfolobus, the genus Saccharolobus, and the genus Aeropyrum.

The enzyme that phosphorylates the 2′ hydroxyl group of RNA may be derived from mesophilic archaea. Mesophilic archaea are also distributed in the phylum Thaumarchaeota. Examples of genera in the phylum Thaumarchaeota include the genus Nitrososphaera and the genus Nitrosopumilus.

Examples of bacteria having an enzyme that phosphorylates the 2′ hydroxyl group of RNA include hyperthermophilic bacteria Aquifex aeolicus (the genus Aquifex) and bacteria belonging to the class Epsilonproteobacteria, which are moderately thermophilic bacteria, for example, Nautilia profundicola (the genus Nautilia), and Leptolyngbya PCC7376 (Leptolyngbya PCC7376, the genus Leptolyngbya).

Examples of mesophilic bacteria having an enzyme that phosphorylates the 2′ hydroxyl group of RNA include bacteria belonging to the phylum Cyanobacteria and the phylum Firmicutes.

The amino acid sequence of the enzyme that phosphorylates the 2′ hydroxyl group of RNA may have one or several amino acid deletions, substitutions or additions compared to sequences shown in each SEQ ID NO. as long as the enzyme has an activity of phosphorylating the 2′ hydroxyl group of RNA. As used herein, “several” means 2 to 10, preferably 2 to 5, and more preferably 2 or 3 although it varies depending on the length of the sequence.

The substituted amino acid is preferably an amino acid that conserves the properties of the original amino acid side chain as classified below. However, the amino acid substitutions are not limited to conservative substitutions.
hydrophobic amino acids (A, I, L, M, F, P, W, Y, V);
hydrophilic amino acids (R, D, N, C, E, Q, G, H, K, S, T);
amino acids having aliphatic side chains (G, A, V, L, I, P);
amino acids having hydroxyl group-containing side chains (S, T, Y);
amino acids having sulfur atom-containing side chains (C, M);
amino acids having carboxylic acid- and amide-containing side chains (D, N, E, Q);
amino acids having base-containing side chains (R, K, H);
amino acids having aromatic-containing side chains (H, F, Y, W)

In one embodiment, the amino acid substitution may be an amino acid which occurs at the corresponding position in the natural enzyme.

The amino acid sequence of each enzyme may consist of an amino acid sequence having 80% or more homology, preferably 80% or more identity to the sequence shown in each SEQ ID NO. as long as the enzyme has an activity of phosphorylating the 2′ hydroxyl group of RNA. The sequence homology or identity is preferably 81%, 82%, 83%, 84%, 85%, 86%, 87%, 88%, 89%, 90%, 91%, 92%, 93%, 94%, 95%, 96%, 97%, 98%, or 99% or more, and more preferably 99% or more.

The amino acid sequences of the enzymes that phosphorylate the 2′ hydroxyl group of RNA are shown in the following SEQ ID NOs. 1 to 8.

Nitrososphaera viennensis EN76
ACCESSION: AIC16789
(SEQ ID NO. 1)
MSQQRASFSGELVLGSPELAKILTYPRGSDKEYAARLAELKGLGVTAVFAGGRTVI
GGTSIAGKGCVGLVVRAKAHGKMCALKIRRMDANRPTMHDEVRYHKIANGAGVGP
RLVGYSDNFMLMEFAEGATIAEWAQGEIDGKQASAVTRSALEQCYALDRAGLDHG
ELSHVDRHIIVSGVQAATTIIDFESASTERKASNVSAAGQSLLVSGAVASALARVLQV
EKEAAIGALKKYKRDQTRENLDAILALVVVA
Nautilia profundicola
ACCESSION: WP_012663809
(SEQ ID NO. 2)
MIIFEKKGTKIRYDIIEKIGEGNRGEVYKAKLEDGRVAAIKWAKNYEIDKEWEILSFLD
GLCAPKPIYRGKRYFIMEYVDGKPLKEYIGSSEYYEVLKKALHNAYVLDEKGVFHGQ
LGRYYHILNTAREVKFIDFERGVFTQNPRNFLQILGYYLFRDEKYDKKALNLIVDLYK
KNRKEALNKIIRLIDES
Leptolyngbya PCC7376
ACCESSION: WP_015134661
(SEQ ID NO. 3)
MAGSQFLTEDQENQLLGYPRISPVEQQARRQELTAFGLEGICQAGEQSLCDLQILG
LGYVGLVVLVIREGKQFALKIRRTNAKRESLLPEAKAIRQANKVGVAPKIYQASDNFI
LMDFIEGKSFLEWLQWAIAKYPTSIILDVINNLLEQAYKLDQIGLDRDDMKCITKDVIV
TATHQPVLLDFSNASGDRRPQNVTALVQGLFWGSVIAKYLKPLLPHCNQEQLLSHL
RHYKGHPNRTNFDTLKQAITLSSLNL
Thermococcus kodakarensis KOD1
ACCESSION: BAD86240
(SEQ ID NO. 4)
MTFEHIISSKRLEGFLRHLAEEGVGGVEPLAKGTTSLVFTGVLGGRKVVIKLQRPDS
PRSNFEKEAELTKIASTFGVTPPIIGLGEFEGLPYLIREFAEGEPILFADVEKEHLFRIV
EKTALLDRLGIDHGQIQGGKHIIIGEDVYLIDFEKAGFRKPNNLTSAMAMIFIGENAIS
KRVREKFGLDEKFREEMKDALRHYKRTGSLSRLLSLLSGL
Sulfurisphaera tokodaii str.7
ACCESSION: BAB65967
(SEQ ID NO. 5)
MRDFIYPRYDENIERELIEHGIKELYSFGPINLGKVNVIGKGKTGIVVLVDDNKVIKIRR
SDSPKETLEIEAKIQIKAFPVAPKIHDYGRNFILMEYIDGRHLTREEKIDIIIDLLRKAKE
LEDKKIEHKELARPYKNVIVKPDRVYIIDYDSASFKENPLNVTSILSWLNFPHLANMY
KKHRDIEEIVSLLYLLNKNQ
Nitrosopumilus maltimus
ACCESSION: ABX13679
(SEQ ID NO. 6)
MAHSFISIKKFVDEPYSEILGYPKSTPRQTKSRINELDKLKIKSISLTGPTTLGKLEILG
KGYVGVVVLA
KKGSREVALKIRRIDSQRKEMKSEAKLLKLVNSVNVGPKMIDVSKNFLVMEYIEGIKI
VDWVNSLKGVGS
VKKLKSTIRKVLEDCYNLDQIGFDHGELSNISKHVIVGKTKSTLIDFESSSVKRRPSN
VTSVTQAIFIGS
GIAKKVQKIYKNPPKEKIIGALKQYKQEKSLENFENLLKILKL
Methanococcus vanielli
ACCESSION: WP_011171556
(SEQ ID NO. 7)
MTNLDFKMNIEGLNAISSKLFDWEILKNLSEYIVFTEYVGKGHRGVVFKAFSDKYIDK
NGNHIILAVKIP
RLDAPKVTIPNEGRILKKTNEFGVGPKVYEYSENHMVMEYVDGEMLKDCIDNLTPE
ELLYVIEETLRQCL
RLDLHKIDHTEIQGGKHIMVSKKGVYIIDFDKAREHSPKNFTSAMSLLFGENYISKKI
MHLLNLSEEKII
LFRKYAKNYKTLFKN
Aquifex aeolicus
ACCESSION: WP_010880344
(SEQ ID NO. 8)
MKFSEFIKEVQNLQELAEGWRGKVYTGYWRGKKVAIKVAKAPDKVKAIQKEAEILE
KLKGLKGFPQILFK
GEDFFVYEFIEGKPFGKLKIGEEEKKRLLKEVLEKAYLLDRMGINRDEFSNIYKNVLV
GDKGEVFVIDFD
RGTFKKNPSNVRQFLQLLKREGFLSQEEAIELGKRYKENPEEVIKEIRKVLS

In one embodiment, the enzyme that phosphorylates the 2′ hydroxyl group of RNA is any one of the following proteins (i) to (iii):
(i) a protein consisting of any one of amino acid sequences SEQ ID NOs. 1 to 8;
(ii) a protein consisting of an amino acid sequence having 80% or more identity to any one of amino acid sequences SEQ ID NOs. 1 to 8 and having an activity of phosphorylating the 2′ hydroxyl group of RNA; and
(iii) a protein consisting of an amino acid sequence in which one or several amino acids are deleted, substituted, or added in any one or several amino acid sequences SEQ ID NOs. 1 to 8 and having an activity of phosphorylating the 2′ hydroxyl group of RNA.

The enzyme that phosphorylates the 2′ hydroxyl group of RNA may be one that is encoded by a base sequence substantially identical to a base sequence that encodes any one of amino acid sequences SEQ ID NOs. 1 to 8, for example, one encoded by a base sequence having one or several base deletions, substitutions or additions compared to each base sequence or a base sequence having 80% or more identity to each base sequence.

A base sequence substantially identical to a base sequence that encodes any one of amino acid sequences SEQ ID NOs. 1 to 8 is one in which a protein, that is encoded by a polynucleotide that has a sequence having 80% or more homology, preferably 80% or more identity to a base sequence shown in any one of SEQ ID NOs. 1 to 8, for example, 81%, 82%, 83%, 84%, 85%, 86%, 87%, 88%, 89%, 90%, 91%, 92%, 93%, 94%, 95%, 96%, 97%, 98%, 99% or more, preferably 99% or more identity, or a polynucleotide that can hybridize with a polynucleotide having a sequence complementary to a base sequence shown in any one of SEQ ID NOs. 1 to 8 under stringent condition, phosphorylates the 2′ hydroxyl group of RNA.

Here, the stringent conditions are hybridization conditions that can be easily determined by those skilled in the art and are generally empirical experimental conditions that depend on the base length of the nucleic acid, the washing temperature, and the salt concentration. Generally, when the base is longer, the temperature for proper annealing is higher, and when the base is shorter, the temperature is lower. Hybrid formation generally depends on the ability of complementary strands to reanneal in an environment slightly below their melting temperature.

The phosphorylation activity of the enzyme that phosphorylates the 2′ hydroxyl group of RNA may be ATP-dependent or GTP-dependent. The phosphorylation activity is preferably ATP-dependent. As used herein, “ATP-dependent” or “GTP-dependent” means that ATP or GTP is substantially used as a phosphate donor in the phosphorylation reaction, and ADP or GDP is generated as a by-product. However, the enzyme that phosphorylates the 2′ hydroxyl group of RNA can also be used with UTP, CTP, pyrophosphate and the like in addition to ATP and GTP.

In one embodiment, the enzyme that phosphorylates the 2′ hydroxyl group of RNA is ArkI as a U^{p} modification enzyme (writer) or its homologue.

ArkI is similar to protein kinases, but has the following characteristic differences.

1. D in the HRD Motif, which is Conserved in Protein Kinases, is Substituted with Q or E

The HRD motif is involved in protein recognition, and particularly, D in this region is very well conserved (https://www.science.org/doi/10.1126/science. 1075762, etc.), but in ArkI, substitution with Q or E provides a motif that could be called an Hx(Q/E) motif. This is a clear difference between the protein kinases and ArkI (H130 to Q132 in Thermococcus ArkI).

2. G in the DFG Motif is Substituted with E

The DFG motif is also well conserved in protein kinases, and is required for retention of metal ions required for the reaction and for activation of the enzyme, but in ArkI, the last G is substituted with E or D, which provides something called a DF (E/D) motif (D149 to E151 in Thermococcus ArkI).

### 3. Original YKR Motif

The C terminal of ArkI has one called a YK (R/K) motif (Y200 to R202 in Thermococcus ArkI), which has a positive charge. The protein kinases do not have such a motif, and it is conceivable that this may contribute to RNA binding.

### 4. Disappearance of Subdomains VIII and X

ArkI does not have subdomains VII and X, which are said to be involved in control and substrate recognition in protein kinases.

It is known that ArkI recognizes tRNA with a variable loop length of 5 bases among tRNAs, and phosphorylates a site called position 47 in the variable loop. tRNA has a characteristic three-dimensional structure, but in tRNA with a variable loop of 5 bases, position 47 partially protrudes outward tRNA as a single strand. It is thought that ArkI recognizes and phosphorylates this site, and thus thermostability and RNA degradation resistance are imparted to tRNA. tRNA targeted by ArkI can be phosphorylated regardless of whether the base at position 47 and the surrounding bases (position 46-48) are U, A, C, or G as long as it is a standard tRNA with a variable loop length of 5 bases. The nucleotide targeted by ArkI may be any nucleotide, but is preferably one having uridine or guanosine as a base.

From the analysis of enzyme reaction kinetics, it can be inferred that phosphorylation modification of tRNA is reversibly adjusted according to the energy state (ATP concentration) of cells. That is, it is thought that ArkI, which performs phosphorylation modification of tRNA, stabilizes tRNA and activates protein synthesis when the energy state is good according to changes in the environment.

### (Stabilizing Agent)

In a second embodiment, there is provided a nucleic acid stabilizer containing an enzyme that phosphorylates the 2′ hydroxyl group of RNA as an active ingredient.

Nucleic acids such as RNA are denatured or degraded when exposed to environmental factors such as heat and oxygen. In particular, RNA is constantly exposed to threat of degradative enzymes in cells. As used herein, “stabilization” refers to a state in which the structure of RNA is stabilized compared to RNA having a 2′ hydroxyl group. Stabilization includes stabilization against heat and resistance to nuclease. For example, when the temperature rises, the three-dimensional structure of tRNA collapses and denaturation occurs, but in the case of tRNA with a phosphorylation modification, the hydrophilic phosphate group protrudes from the center of tRNA to the outside (solvent side), and it is possible to prevent rotation of the RNA main chain, and therefore, to prevent collapse of the three-dimensional structure. In addition, when tRNA is phosphorylated, its heat resistance increases, and degradation with RNase becomes slow. Heat resistance can be evaluated according to an increase in the melting temperature.

The stabilization may be a significant level of improvement compared to RNA having a 2′ hydroxyl group, and may be an improvement of stability of at least about 1%, 5%, 25%, 50%, 75%, 100%, 125%, 150%, 175%, 200%, 250%, 300%, 350%, 400%, 450%, 500%, 600%, 700%, 800%, 900%, or 1,000% or more.

The enzymes that phosphorylate the 2′ hydroxyl group of RNA as an active ingredient are as described above. Among them, ArkI can increase the thermostability by several degrees or more, for example, by more than 5° C., when the U^{p}47 modification is introduced into tRNA. RNA to be phosphorylated may contain known modified bases. For example, some modified bases are important for formation and stabilization of the higher-order structure of RNA, and a sulfur modification has a role of stabilizing the three-dimensional structure. For example, in almost all tRNA species in mesophilic organisms, position 54 is 5-methyl uridine (m⁵U), but some of the positions are sulfurized in tRNA of thermophilic bacteria and the like to form 5-methyl-2-thiouridine (m⁵s²U). In addition to sulfurization, as modified bases that contribute to stability, Cm32 and the like that contribute to stabilization of the anticodon/loop structure and strengthening of anticodon/codon pairing are known.

The stabilizer may contain other ingredients that are known to stabilize nucleic acids as long as they do not impair the phosphorylation activity of the active ingredient. Polyamines such as spermine are known to bind to the secondary structure of RNA such as tRNA, mRNA, and rRNA, and contribute to stabilizing their conformation. In addition, the 5′ cap structure in the tRNA precursor has a role of protecting it from degradation by 5′ exonuclease, and an enzyme such as RNA polymerase that contributes to formation of the 5′ cap structure may be added to the stabilizer.

RNA to be treated with the stabilizer is any RNA or RNA contained in a sample. The sample may be a biological sample. The biological sample may be any sample containing RNA. Such RNA includes RNA isolated, secreted or released from a living organism. Examples of biological samples include cells, tissues, body fluids (blood, etc.), feces and urine, secretions (saliva, etc.) collected from living organisms, and secretions on the skin of living organisms.

### (Vector)

In a third embodiment, there is provided a vector containing a nucleic acid encoding an enzyme that phosphorylates the 2′ hydroxyl group of RNA.

An expression vector containing a polynucleotide encoding an enzyme that phosphorylates the 2′ hydroxyl group of RNA can be obtained by a method known to those skilled in the art. The expression vector preferably contains a promoter, a terminator and the like suitable for expressing the enzyme that phosphorylates the 2′ hydroxyl group of RNA. The expression vector may further contain other appropriate control sequences such as an enhancer.

The vector can be appropriately selected from among expression vectors well known to those skilled in the art in consideration of the type of host cells, the expression efficiency of the enzyme, and the like, and the type of the vector may be a plasmid vector or a virus vector derived from a retrovirus or adenovirus.

The host cells are not limited as long as the cells can express an enzyme from an expression vector, and may be prokaryotic cells such as E. coli, mammal-derived cells such as CHO, COS, NIH3T3, HEK293, HEK293T, and COS-7, insect-derived cells such as F9, or eukaryotic cells such as yeast cells. Examples of mammals include primates such as humans and chimpanzees, and rodents such as mice, rats, and hamsters.

Transfection of an expression vector into host cells can be performed using, for example, methods well known to those skilled in the art such as an electroporation method, a calcium phosphate method, a lipofection method, and a DEAE dextran method, or methods using commercially available reagents.

Host cells (transfectants) transfected with an expression vector containing a polynucleotide encoding an enzyme that phosphorylates the 2′ hydroxyl group of RNA include:
a transfectant that contains the expression vector and expresses an enzyme that phosphorylates the 2′ hydroxyl group of RNA; and
a transfectant (stable transfectant) that expresses an enzyme that phosphorylates the 2′ hydroxyl group of RNA by incorporating a polynucleotide encoding an enzyme that phosphorylates the 2′ hydroxyl group of RNA into the chromosome of host cells in an expressible manner. Such stable transfectants may or may not contain an expression vector containing a polynucleotide encoding an enzyme that phosphorylates the 2′ hydroxyl group of RNA. The transfectant is preferably a mammal-derived cell such as CHO, COS, NIH3T3, HEK293, HEK293T, or COS-7.

### (Kit)

In a fourth embodiment, there is provided a kit comprising
1) an enzyme that phosphorylates the 2′ hydroxyl group of RNA;
2) a nucleic acid stabilizer containing an enzyme that phosphorylates the 2′ hydroxyl group of RNA as an active ingredient; or
3) a vector containing a nucleic acid encoding an enzyme that phosphorylates the 2′ hydroxyl group of RNA.

The kit may further include optional ingredients depending on the application of an enzyme that phosphorylates the 2′ hydroxyl group of RNA.

In one embodiment, the kit further includes a nucleic acid and optionally ATP.

In one embodiment, the kit further includes an enzyme that dephosphorylates the 2′ phosphate group of RNA, optionally, the 2′ phosphate group of RNA or a nucleic acid containing the 2′ phosphate group of RNA, and nicotinamide adenine dinucleotide (NAD⁺).

The amino acid sequences of the enzymes that dephosphorylate the 2′ hydroxyl group of RNA are shown in the following SEQ ID NOs. 112 to 114.

Escherichia coli (strain K12)
ACCESSION: NP_418751
(SEQ ID NO. 112)
MAKYNEKELADTSKFLSFVLRHKPEAIGIVLDREGWADIDKLILCAQKAGKRLTRALL
DTVVATSDKKRFSYSSDGRCIRAVQGHSTSQVAISFAEKTPPQFLYHGTASRFLDEI
KKQGLIAGERHYVHLSADEATARKVGARHGSPVILTVKAQEMAKRGLPFWQAENG
VWLTSTVAVEFLEW
Thermococcus kodakarensis
ACCESSION: WP_011249257
(SEQ ID NO. 113)
MKPERKRVSKLMAYILRHSPEEFGLRPDVEGFVSLNELVNALKTVYPEVTEEFVREI
VENDPKGRYEIRGDRIRARYGHSFPVSLDHEEDTESRFLYHGTPRRNLPSILKEGLK
PMKRQYVHVSTDKIEALETGRRHGREVVLLVIDAECLRKRGFKIYKAGKNVRIVERV
PPDCITLAV
Saccharomyces cerevisiae
ACCESSION: CAA99116
(SEQ ID NO. 114)
MRQVLQKDKRDVQLSKALSYLLRHTAVKEKLTIDSNGYTPLKELLSHNRLKTHKCTV
DDIHRIVKENDKQ
RFHIKTLGADEEWICATQGHSIKSIQPSDEVLVPITEASQLPQELIHGTNLQSVIKIIES
GAISPMSRNH
VHLSPGMLHAKGVISGMRSSSNVYIFIDCHSPLFFQTLKMFRSLNNVYLSSSIPVELI
QKVVVKGNLKDE
EKLDTLRRILHERNIPLEKI

Reversible modifications of RNA include phosphorylation and dephosphorylation. Examples of enzymes that phosphorylate the 2′ hydroxyl group of RNA include ArkI and homologues thereof, and examples of enzymes that dephosphorylate the 2′ phosphate group of RNA obtained using such enzymes include members of the KptA/Tpt1 family and homologues thereof.

KptA and Tpt1 belong to the ADP-ribosyltransferase family. The characteristic sequence of KptA/Tpt1 is an H-H-h (the lower case h is a hydrophobic amino acid) motif (H98/H122/V165 in the case of TkKptA), and this is a partial sequence that is well conserved within the ADP-ribosyltransferase family when broadly aligned (https://www.sciencedirect.com/science/article/pii/S2001037021001355#f0010).

The amino acid sequence of the enzyme that dephosphorylates the 2′ phosphate group may consist of an amino acid sequence having 80% or more homology, preferably 80% or more identity to the amino acid sequence of members of the KptA/Tpt1 family or homologues thereof as long as the enzyme has an activity of dephosphorylating the 2′ phosphate group of RNA. The sequence homology or identity is preferably 81%, 82%, 83%, 84%, 85%, 86%, 87%, 88%, 89%, 90%, 91%, 92%, 93%, 94%, 95%, 96%, 97%, 98%, or 99% or more, and more preferably 99% or more.

The enzyme that dephosphorylates the 2′ phosphate group of RNA may be one that is encoded by a base sequence substantially identical to a base sequence that encodes an amino acid sequence of a member of the KptA/Tpt1 family or a homologue thereof, for example, one encoded by a base sequence having one or several base deletions, substitutions or additions compared to each base sequence or a base sequence having 80% or more identity to each base sequence.

A base sequence substantially identical to a base sequence that encodes an amino acid sequence of a member of the KptA/Tpt1 family or a homologue thereof is one in which a protein, that is encoded by a polynucleotide having a sequence having 80% or more homology, preferably 80% or more identity, for example, 81%, 82%, 83%, 84%, 85%, 86%, 87%, 88%, 89%, 90%, 91%, 92%, 93%, 94%, 95%, 96%, 97%, 98%, 99% or more, preferably 99% or more identity to a base sequence that encodes an amino acid sequence of a member of the KptA/Tpt1 family or a homologue thereof, or a polynucleotide that can hybridize with a polynucleotide having a sequence complementary to a base sequence that encodes an amino acid sequence of a member of the KptA/Tpt1 family or a homologue thereof under stringent conditions, dephosphorylates the 2′ phosphate group of RNA.

In one embodiment, the enzyme that dephosphorylates the 2′ phosphate group of RNA is KptA.

In one embodiment, KptA is any one of the following proteins (i) to (iii):
(i) a protein consisting of any one of amino acid sequences SEQ ID NOs. 112 to 114;
(ii) a protein consisting of an amino acid sequence having 80% or more identity to any one of amino acid sequences SEQ ID NOs. 112 to 114 and having an activity of dephosphorylating the 2′ phosphate group of RNA; and
(iii) a protein having an amino acid sequence in which one or several amino acids are deleted, substituted, or added in any one or several amino acid sequences SEQ ID NOs. 112 to 114 and having an activity of dephosphorylating the 2′ phosphate group of RNA.

In one embodiment, the enzyme that dephosphorylates 2′ phosphorylated uridine is Tpt1p or a homologue thereof.

In one embodiment, the kit further includes tRNA.

### (Composition)

In a fifth embodiment, there is provided a composition comprising
1) an enzyme that phosphorylates the 2′ hydroxyl group of RNA;
2) a nucleic acid stabilizer containing an enzyme that phosphorylates the 2′ hydroxyl group of RNA as an active ingredient; or
3) a vector containing a nucleic acid encoding an enzyme that phosphorylates the 2′ hydroxyl group of RNA.

The composition can be used for various applications, for example, for medicinal applications, and may further contain optional ingredients depending on the application. Examples of medicinal applications include treatment or prevention of tRNA-related diseases.

In one embodiment, the tRNA-related disease is a disease caused by reduced or deficient 5-taurinomethyluridine (τm⁵U) modification in mitochondrial tRNA.

The τm⁵U (5-taurinomethyluridine) modification is a modification of mitochondrial tRNA, and is generally found in a wide range of animals from vertebrates (humans, mice, cows, cats, etc.), protochordata (sea squirts) to mollusks (squids). As used herein, the “τm⁵U modification” is a taurine modification of mitochondrial tRNA^{Leu(UUR)} and is a post-transcriptional modification in which a taurinomethyl group is bound to position 5 of a uracil base located at the wobble-position (position 34) corresponding to the first letter of the anticodon (UAA) of tRNA^{Leu (UUR)}.

Deficient or reduced mitochondrial tRNA modifications often cause diseases. Mitochondrial encephalomyopathy (MELAS) characterized by apoplexy and myoclonus epilepsy associated with ragged-red fibers (MERRF) characterized by epilepsy are thought to be caused by point mutations in tRNA genes, and in these mutant mitochondrial tRNAs, taurine modification significantly decreases, and uridine remains unmodified.

In one embodiment, the disease caused by reduced or deficient τm⁵U modification is mitochondrial encephalomyopathy (MELAS).

Reduced taurine modification can be evaluated by the rate of τ⁵U modification at position 34 (also called a τm⁵U modification rate) contained in a specific tRNA called mitochondrial tRNA^{Leu(UUR)}. As used herein, the “τm⁵U modification rate” can be represented by the following formula.

$$τ {m}^{5} U modification rate ( \% ) = τ {m}^{5} U / ( τ {m}^{5} U + U ) \times 100$$

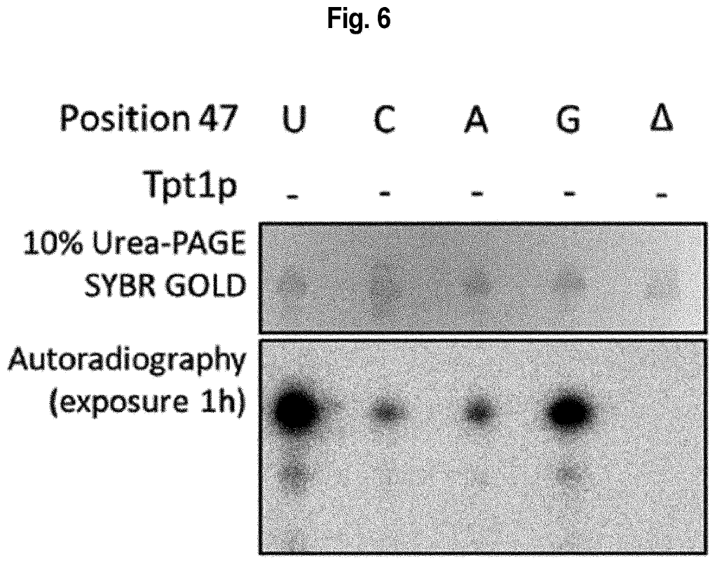

When a reverse transcription reaction is performed on mitochondrial tRNA^{Leu(UUR)}, the reaction proceeds according to the flow shown in FIG. 6 in WO2023/282306, and in the case of τm⁵U modification, the reverse transcription reaction stops at a position corresponding to position 33 of mitochondrial tRNA^{Leu(UUR)} (center in FIG. 7 in the same publication). However, when a reverse transcription reaction is performed using mitochondrial tRNA^{Leu(UUR)} as a template in the presence of a water-soluble carbodiimide such as CMC, τm⁵U derivatized due to contact with a water-soluble carbodiimide causes the reverse transcription reaction to be stopped at a position corresponding to position 35 (right in FIG. 7 in the same publication). On the other hand, the unmodified U undergoes a reverse transcription reaction, dideoxyguanosine triphosphate (ddGTP) is introduced into C at position 32, and the elongation reaction is terminated (left in FIG. 7 in the same publication). The entire contents of the above publication are incorporated herein by reference.

When the extension reaction product is quantified by denature polyacrylamide gel electrophoresis or the like, the band intensities at position 32, position 33, and position 35 are quantified, a calibration curve can be created between the band intensity ratio calculated from the following formula and the τm⁵U modification rate calculated from the sample mixing ratio, and the band intensity ratio can be correlated with the τm⁵U modification rate.

Band intensity ratio=(band intensity at position 33+band intensity at position 35)/(total value of band intensities at position 32, position 33, and position 35)

As used herein, “reduced τm⁵U modification” means the τm⁵U modification rate is reduced to a degree at which diseases such as MELAS and MERRF are caused compared to normal RNA^{Leu(UUR)} in which taurine modification is formed. For example, the τm⁵U modification rate in myoblasts derived from a certain MELAS patient is less than 20%. However, the τm⁵U modification rate varies depending on cells, tissues, and patients. For example, it has been found that the τm⁵U modification rate in Hela cells, which are generally used cells derived from human cervical cancer, is 96.3%.

Examples of diseases caused by reduced or deficient τm⁵U modification include mitochondrial encephalomyopathy such as MELAS and MERRF, and the present invention is not limited to these diseases.

In order to treat or prevent diseases caused by reduced or deficient τm⁵U modification, an excess amount of the taurine modification enzyme MTO1 is administered to a patient or MTO1 is overexpressed in a patient to whom a nucleic acid encoding MTO1 is administered.

As used herein, an “excess amount” of MTO1 or “overexpression” of a nucleic acid encoding MTO1 refers to a level that is higher compared to a control, for example, higher than the amount of MTO1 present in the patient's body before the pharmaceutical composition is administered, and refers to a state that is at least about 1%, 5%, 25%, 50%, 75%, 100%, 125%, 150%, 175%, 200%, 250%, 300%, 350%, 400%, 450%, 500%, 600%, 700%, 800%, 900%, 1,000% or more greater than the control. 500% to 1,000% or more is preferable. In a preferable aspect, the excess amount and overexpression refer to a level at which, when MTO1 or a nucleic acid encoding MTO1 is administered to a patient, the τm⁵U modification rate is improved compared to before administration. Therefore, when MTO1 is directly administered, the excess amount is appropriately determined depending on patients and desired effects.

Overexpression of MTO1 can be achieved by methods well known to those skilled in the art. For example, overexpression may be achieved by introducing a nucleic acid encoding MTO1 into cells of a patient using, for example, a gene introduction system using a lentivirus or retrovirus, and expressing the nucleic acid. When gene expression is performed using a virus gene introduction vector, the gene may be operably linked downstream of an appropriate promoter, inserted into a gene introduction vector and introduced into cells to express the target gene.

MTO1 used when an excess amount of MTO1 is administered may be a protein that is considered as a fragment thereof or modified MTO1 as long as it has a function of a desired MTO1.

When a nucleic acid encoding MTO1 is administered to a patient to overexpress MTO1 in the body, the nucleic acid administered may be any nucleic acid as long as it can express a protein having a desired function of MTO1. A vector containing a nucleic acid encoding MTO1 can be prepared by a well-known method as a vector system that can express a protein having a desired function of MTO1.

The term “operably” linked means that a promoter, a gene encoding MTO1 and the like are linked so that desired expression of the gene is achieved by the promoter. Any known promoter can be used. In addition, the promoter may be genetically modified to increase transcription of a gene encoding MTO1.

When the nucleic acid encoding MTO1 is contained in a vector, the vector may be a virus vector or a plasmid. The virus vector may be selected from the group consisting of known vectors, for example, a retrovirus vector, a lentivirus vector, an adenovirus vector and an adeno-associated virus vector. The vector may be an autonomously replication vector, for example, a plasmid, an extrachromosomal element, a minichromosome, or an artificial chromosome.

When an excess amount of MTO1 is administered to a patient or when MTO1 is overexpressed in a patient to whom a nucleic acid encoding MTO1 is administered, the τm⁵U modification rate is significantly improved. However, such an improvement in the τm⁵U modification rate is thought to exceed an improvement in the m⁵U modification rate due to over administration of GTPBP3 which constitute the τm⁵U modification enzyme together with MTO1 or overexpression of a nucleic acid encoding GTPBP3.

The formulation, dosage form, and dosage of MTO1 or a nucleic acid encoding MTO1 can be determined according to those generally known for polymeric drugs or nucleic acid drugs. For example, the formulation may be a parenteral preparation form such as an injection or a drop. In addition, examples of carriers that can be used in such parenteral preparations include aqueous carriers such as a physiological saline and an isotonic solution.

The pharmaceutical composition may contain ingredients such as a pharmaceutically acceptable buffer, a stabilizer, a preservative, and other additives. The pharmaceutically acceptable ingredients are well known to those skilled in the art, and those appropriately selected from among, for example, ingredients described in the specification of Japanese Pharmacopoeia 17th Edition according to the form of preparation within the scope of their ordinary practical capabilities can be used.

The pharmaceutical composition can be administered orally or parenterally (topically, rectally, intravenously, intraarterially, intramuscularly, or subcutaneously). The administration method is not particularly limited, and whole body administration by injection or dripping is preferable, and examples thereof include intravenous administration and intraarterial administration. In another aspect, administration to the periphery related to the central nervous system (CNS) is also preferable. In order to curb apoplexy in MELAS, it is preferable to introduce the pharmaceutical composition into vascular endothelial cells in the brain. It is more preferable to administer it to skeletal muscles throughout the body.

In another aspect, there is provided a method for treating or preventing a disease caused by reduced or deficient τm⁵U modification in mitochondrial tRNA, comprising a step of administering MTO1 or a nucleic acid encoding MTO1 to a subject, wherein an excess amount of MTO1 is administered or MTO1 is overexpressed in a patient to whom the nucleic acid is administered.

Such a method may be performed in combination with other treatment methods used to treat or prevent a disease caused by reduced or deficient τm⁵U modification. For example, combinations of taurine, taurine chloramine, taurine precursors, tauroursodeoxycholic acid (TUDCA) and the like, which are known as treatment agents for MELAS, with the use of the pharmaceutical composition are conceivable. Among these, a high-dosage taurine supplementation therapy is preferable because its effectiveness in curbing the recurrence of strokelike episodes in MELAS has been confirmed. MTO1 or a nucleic acid encoding MTO1 may be administered simultaneously with other active ingredients such as taurine, or may be administered at different times. When administered simultaneously, the active ingredients may be contained together in a pharmaceutical composition.

The subjects to which MTO1 or a nucleic acid encoding MTO1 is administered are not limited to humans, and the scope of subjects includes non-human vertebrates (monkeys, mice, rats, hamsters, guinea pig, rabbits, cats, dogs, swine, cows, horses, sheep, birds, reptiles, amphibians, fish, etc.) which can suffer from diseases caused by reduced or deficient τm⁵U modification, protochordata, mollusks and the like.

In place of administration of MTO1 or a nucleic acid encoding MTO1, or together with administration of MTO1 or a nucleic acid encoding MTO1, a treatment to enhance expression of MTO1 in the patient can also be performed. For example, enhanced expression of MTO1 can be induced by enhancing the subject's own endogenous MTO1 expression activity.

### Modification Method

In a sixth embodiment, there is provided a method for modifying the 2′ hydroxyl group of RNA, comprising a step of bringing RNA into contact with an enzyme that phosphorylates the 2′ hydroxyl group of RNA.

In recent years, it has become clear that, in eukaryotes, post-transcriptional mRNA undergoes methylation modification, including N6-methyladenosine (m⁶A), at various sites. These modifications affect the stability and translation efficiency of individual mRNA. In addition, the presence of demethylases that remove these methylation modifications has been clearly found, and the concept of epitranscriptome, which changes the methylation modification rate in a spatiotemporal manner in response to the environment to control gene expression and cell activity, has been proposed.

N6-methyladenosine (m⁶A) has an important role in RNA metabolism, and functions as a reversible RNA modification in eukaryotic mRNAs and non-coding

RNAs. When an enzyme that phosphorylates the 2′ hydroxyl group of RNA and an enzyme that dephosphorylates the 2′ phosphate group of RNA are used in combination, reversible modification of RNA becomes possible. Particularly, reversible modification of U^{p}47, which is involved in thermostability, is thought to be beneficial for hyperthermophilic organisms in very harsh environments.

Reversible modifications of RNA include phosphorylation and dephosphorylation. Examples of enzymes that phosphorylate the 2′ hydroxyl group of RNA include ArkI and homologues thereof, and examples of enzymes that dephosphorylate the 2′ phosphate group of RNA obtained using such enzymes include members of the KptA/Tpt1 family and homologues thereof.

### (Stabilization Method)

In a seventh embodiment, there is provided a method for stabilizing a nucleic acid, comprising a step of bringing a nucleic acid into contact with an enzyme that phosphorylates the 2′ hydroxyl group of RNA.

The nucleic acid containing uridine is not particularly limited, and a nucleic acid for which enhanced stability is required, for example, RNA, particularly tRNA, is preferable. The definition of stabilization of nucleic acids is as described above, and examples of stabilization imparted to nucleic acids include thermostability and resistance to RNase.

The nucleic acid containing uridine may be brought into contact with a substance other than the enzyme that phosphorylates the 2′ hydroxyl group of RNA. For example, an enzyme that phosphorylates the 2′ hydroxyl group of RNA may be used in combination with a known stabilization ingredient that stabilizes a nucleic acid containing uridine. The stabilization method may include any step other than the contact step.

### (Production Method)

In an eighth embodiment, there is provided a method for producing 2′ phosphorylated uridine or a nucleic acid containing 2′ phosphorylated uridine, comprising a step of bringing uridine or a nucleic acid containing uridine into contact with an enzyme that phosphorylates the 2′ hydroxyl group of RNA.

The 2′ phosphorylated uridine or a nucleic acid containing 2′ phosphorylated uridine is not particularly limited, and RNA, particularly tRNA is preferable.

The nucleic acid containing uridine may be brought into contact with a substance other than the enzyme that phosphorylates the 2′ hydroxyl group of RNA. For example, an enzyme that phosphorylates the 2′ hydroxyl group of RNA may be used in combination with a known ingredient used in the production of 2′ phosphorylated uridine or a nucleic acid containing 2′ phosphorylated uridine. The production method may include any step other than the contact step.

Hereinafter, the present invention will be described in more detail with reference to examples, but the present invention is not limited thereto.

### EXAMPLES

### 1. Archaeal Strains and Media

Sulfurisphaera tokodaii str. 7, Methanosarcina acetivorans C2A, and Thermoplasma acidophilum were provided from Dr. Tairo Oshima (Kyowa Kako Co., Ltd.), Dr. Takashi Yokokawa (Gifu University), and Dr. Hiroyuki Hori (Ehime University), respectively. Sulfolobus acidocaldarius (JCM no. 8929), Saccharolobus solfataricus (JCM no. 8930), Aeropyrum pernix (JCM no. 9820), Pyrobaculum oguniense (JCM no. 10595) and N. viennensis (JCM no. 19564) were obtained from Japan Collection of Microorganisms, RIKEN BRC which is participating in the National BioResource Project of the MEXT, Japan.

S. tokodaii and S. acidocaldarius were cultured at 80° C. in JCM medium no. 165 consisting of 1 g/L yeast extract, 1 g/L casamino acids, 1.3 g/L (NH₄)₂SO₄, 0.28 g/L KH₂PO₄, 0.25 g/L MgSO₄-7H₂O, 0.07 g/L CaCl₂-2O, 2.0 mg/L FeCl₃-6H₂O, 1.8 mg/L MnCl₂-4H₂O, 4.5 mg/L Na₂B₄O₇-10H₂O, 0.22 mg/L ZnSO₄-7H₂O, 0.05 mg/L CuCl₂-2H₂O, 0.03 mg/L Na₂MoO₄-2H₂O, 0.03 mg/L VOSO₄—H₂O, and 0.01 mg/L CoSO₄-7H₂O (adjusted to pH 2.5 with H₂SO₄). S. solfataricus was cultured at 80° C. in JCM medium no. 171 consisting of 1 g/L yeast extract, 2.5 g/L (NH₄)₂SO₄, 3.1 g/L KH₂PO₄, 0.2 g/L MgSO₄-7H₂O, 0.25 g/L CaCl₂-2H₂O, 1.8 mg/L MnCl₂-4H₂O, 4.5 mg/L Na₂B₄O₇-10H₂O, 0.22 mg/L ZnSO₄-7H₂O, 0.05 mg/L CuCl₂-2H₂O, 0.03 mg/L Na₂MoO₄-2H₂O, 0.03 mg/L VOSO₄—H₂O, and 0.01 mg/L CoSO₄-7H₂O (adjusted to pH 4.0 with H₂SO₄). A. pernix was cultured at 90° C. in JCM medium no. 224 consisting of 1 g/L yeast extract, 1 g/L peptone, 1 g/L Na₂S₂O₃-5H₂O, 24.0 g/L NaCl, 7.0 g/L MgSO₄-7H₂O, 5.3 g/L MgCl₂-6H₂O, 0.7 g/L KCl, and 0.1 g/L CaCl₂-2H₂O (adjusted to pH 7.0 with NaOH). P. oguniense was cultured at 90° C. in JCM medium no. 165 with addition of 1.0 g/L Na₂S₂O₃-5H₂O (adjusted to pH 7.25 with NaOH). N. viennensis was cultured at 42° C. in JCM medium no. 1004 consisting of 1 g/L NaCl, 0.5 g/L KCl, 0.4 g/L MgCl₂-6H₂O, 0.2 g/L KH₂PO₄, 0.1 g/L CaCl₂-2H₂O, 1.0 mL/L modified trace element mixture (30 mg/L H₃BO₃, 100 mg/L MnCl₂-4H₂O, 190 mg/L CoCl₂-6H₂O, 24 mg/L NiCl₂-6H₂O, 2 mg/L CuCl₂-2H₂O, 144 mg/L ZnSO₄-7H₂O, 36 mg/L Na₂MoO₄-2H₂O, and 0.3% HCl), 1.0 mL/L vitamin solution (20 mg/L biotin, 20 mg/L folic acid, 100 mg/L pyridoxine-HCl, 50 mg/L thiamine-HCl, 50 mg/L riboflavin, 50 mg/L nicotinic acid, 50 mg/L DL-calcium pantothenate, 1 mg/L vitamin B₁₂, 50 mg/L p-aminobenzoic acid, and 2 g/L choline chloride (adjusted to pH 7.0 with KOH)), 1.0 mL/L 7.5 mM EDTA-Na—Fe (III) solution (pH 7.0), 2.0 mL/L 1 M NaHCO₃ solution, 10 mL/L HEPES solution (238.4 g/L HEPES (free acid) and 24 g/L NaOH), 1.0 mL/L 1 M NH₄Cl solution, and 1.0 mL/L 1 M sodium pyruvate solution (adjusted to pH 7.6 with NaOH).

Thermococcus kodakarensis was cultured under anaerobic conditions at 83° C., 87° C. or 91° C. in a rich medium (ASW-YT-S° or MA-YT-Pyr) or a synthetic medium containing amino acids (ASW-AA-S°). The ASW-YT-S° medium was composed of 0.8×artificial sea water (ASW) (Sato, T., Fukui, T., Atomi, H., and Imanaka, T. (2003). Targeted gene disruption by homologous recombination in the hyperthermophilic archaeon Thermococcus kodakaraensis KOD1. J Bacteriol 185, 210-220. 10.1128/jb. 185.1.210-220.2003), 10 g/L yeast extract, 5.0 g/L tryptone, 2.0 g/L elemental sulfur, and 0.1% (wt/vol) resazurin. The MA-YT-Pyr medium was composed of 30.5 g/L Marine Art SF-1 (Osaka Yakken, Osaka, Japan), 10 g/L yeast extract, 5.0 g/L tryptone, 5.0 g/L pyruvate sodium, and 0.1% (wt/vol) resazurin. The ASW-AA-S⁰ medium was composed of 0.8×ASW, 0.5×amino acids solution (Sato et al., 2003), modified Wolfe's trace minerals (0.5 g/L MnSO₄-2H₂O, 0.1 g/L CoCl₂, 0.1 g/L ZnSO₄, 0.01 g/L CuSO₄-5H₂O, 0.01 g/L AlK(SO₄)₂, 0.01 g/L H3BO₃, and 0.01 g/L NaMoO₄-2H₂O), 5.0 mL/L vitamin mixture (Robb, F. T. & Place, A. R. Media for Thermophiles. (Cold Spring Harbor Laboratory Press, 1995), 2.0 g/L elemental sulfur, and 0.1% (wt/vol) resazurin. For plate culture, a 2.0 mL/L polysulfide solution (67% Na₂S-9H₂O solution containing 20% elemental sulfur) was added in place of elemental sulfur, and 1.0% Gelrite (FUJIFILM Wako Pure Chemical Corporation) was added to the medium and solidified. When pyrF gene-deficient strains were selected, 0.75% 5-fluoroorotic acid (5-FOA) was added to the medium. The ASW-YT-S° medium was used for general culture, MA-YT-Pyr was used for growth comparison, and the ASW-AA-S⁰ medium was used for constructing gene-deficient strains.

2. Preparation of tRNA Fractions

For small-scale preparation (˜100-ml culture), archaeal cells were resuspended in 3 ml solution D (4 M guanidine thiocyanate, 25 mM citrate-NaOH (pH 7.0), 0.5% (wt/vol) N-lauroylsarcosine sodium salt and 1 mM 2-mercaptoethanol) and mixed with an equal volume of water-saturated phenol and 1/10 volume of 3 M sodium acetate (pH 5.3). The mixture was shaken for 1 h on ice and mixed with ⅕ volume of chloroform, followed by centrifugation at 8,000 g for 10 min at 4° C. The supernatant was collected and mixed with an equal volume of chloroform, followed by centrifugation at 8,000 g for 10 min at 4° C. Total RNA was obtained from the resultant supernatant by isopropanol precipitation. The total RNA prepared in this manner was separated by 10% denaturing PAGE, followed by staining with SYBR Gold or toluidine blue. The visualized tRNA fraction including class I and class II tRNAs was cut out and eluted from the gel slice with elution buffer (0.3 M sodium acetate (pH 5.3) and 0.1% (wt/vol) SDS), followed by filtration to remove the gel pieces and ethanol precipitation for RNA-MS analysis of the tRNA fraction.

For large-scale preparation of tRNA fractions from S. tokodaii, cell pellets (53 g) were resuspended in 530 ml solution D and then mixed with 53 ml of 3 M sodium acetate (pH 5.3) and 425 ml neutralized phenol. The mixture was shaken for 1 h on ice to which 106 ml chloroform/isoamyl alcohol (49:1) was added, followed by centrifugation at 4,500 g for 20 min at 4° C. The supernatant was collected and mixed with 106 ml chloroform/isoamyl alcohol (49:1), followed by centrifugation at 4,500 g for 15 min at 4° C. The aqueous phase was collected and then subjected to isopropanol precipitation. The collected RNA was resuspended in 53 ml water and mixed with 80 ml TriPure Isolation Reagent (Roche), followed by centrifugation at 10,000 g for 20 min at 4° C. The supernatant was collected and mixed with 36 ml chloroform/isoamyl alcohol (49:1), followed by centrifugation at 10,000 g for 10 min at 4° C. The aqueous phase was collected and precipitated with isopropanol. The prepared total RNA (608 mg) was dissolved in 250 ml of buffer consisting of 20 mM HEPES-KOH (pH 7.6), 200 mM NaCl and 1 mM DTT and then loaded on a DEAE Sepharose Fast Flow column (320-ml beads) and fractionated with a gradient of NaCl from 200 to 500 mM. Fractions containing tRNA were collected by isopropanol precipitation.

3. Isolation and purification of tRNA

It was very difficult to isolate and purify tRNA from thermophilic organisms because of their high G+C content and high melting temperature due to complex RNA modifications. Thus, the inventors optimized unique RNA isolation and purification methods: reciprocal circulating chromatography (RCC) (Miyauchi, K., Ohara, T. & Suzuki, T. Automated parallel isolation of multiple species of non-coding RNAs by the reciprocal circulating chromatography method. Nucleic Acids Res 35, e24, doi: 10.1093/nar/gkl1129 (2007)) and chaplet column chromatography (CCC) (Suzuki, T. & Suzuki, T. Chaplet column chromatography: isolation of a large set of individual RNAs in a single step. Methods Enzymol 425, 231-239, doi: 10.1016/S0076-6879 (07) 25010-4 (2007)). RCC was performed on a sample containing about 200 A260 units of S. tokodaii tRNA fractions under the following conditions: hybridization was performed using a 6×NHE buffer (30 mM HEPES-KOH (pH7.5), 15 mM EDTA (pH 8.0), 1.2 M NaCl, and 1 mM DTT) at 66° C., washing was performed using a 0.1×NHE buffer (0.5 mM HEPES-KOH (pH 7.5), 0.25 mM EDTA (pH 8.0), 20 mM NaCl, and 0.5 mM DTT) at 50° C., and elution was performed using a 0.1×NHE buffer at 72° C. The eluted tRNA was collected by precipitation with ethanol. Mature tRNA and precursor tRNA were isolated by 10% denaturing PAGE and then stained with SYBR Gold. The visualized mature tRNA and precursor tRNA bands were excised from the gel and eluted with an elution buffer, gel pieces were then removed by filtration through a filter and precipitated with ethanol.

In order to crystallize natural tRNA with U^{p}47, a large amount of S. tokodaii tRNA^{Val3} was isolated and purified by CCC. From 2,000 A260 units of S. tokodaii tRNA fractions, tRNA^{Val3}, tRNA^{Ile2}, and tRNA^{Phe} were isolated and purified by CCC using a tandem affinity chaplet column. The conditions for isolation and purification were as follows: hybridization was performed using a 6×NHE buffer at 72° C., washing was performed using a 0.1×NHE buffer at 50° C. for each column, and elution was performed using a 0.1×NHE buffer at 72° C. The eluted tRNAs were collected by precipitation with isopropanol. Table 1 shows the base sequences of the DNA probes used for isolation and purification. In addition, tRNA^{Val3} isolated as described below was additionally purified by anion exchange chromatography and thus tRNA^{Val2} was completely removed.

### 4. RNA Mass Spectrometry (RNA-MS)

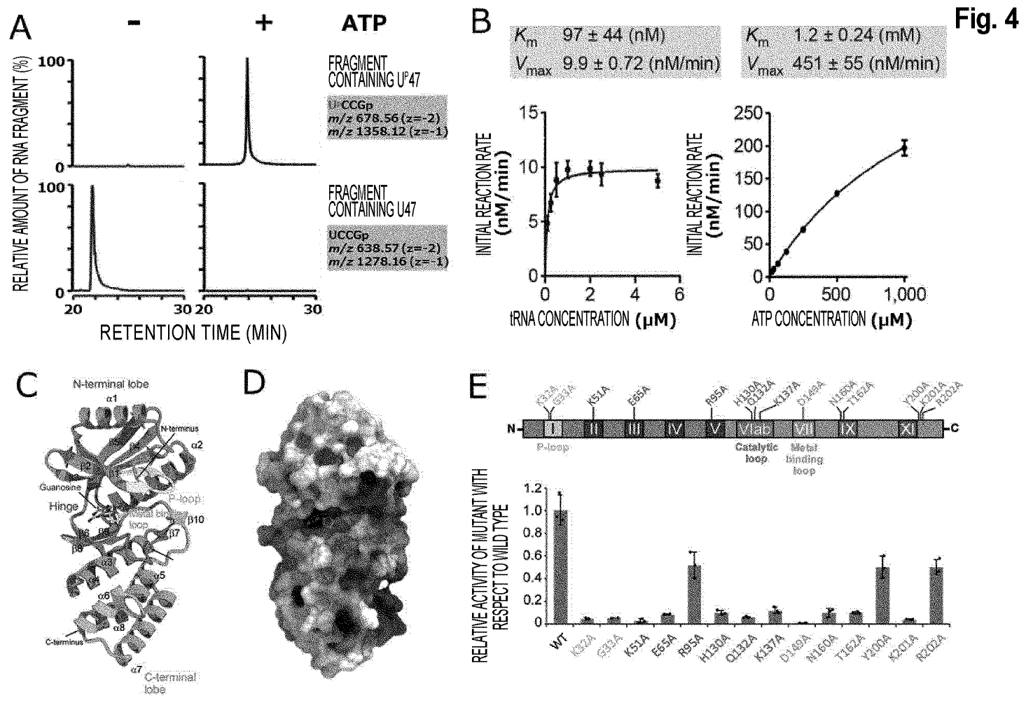

In order to analyze tRNA fragments by RNA-MS, 30 ng (900 fmol) of the isolated and purified tRNA or 150 ng (4.5 μmol) of tRNA fractions were digested with RNase T1 (Epicentre or Thermo Fisher Scientific) or RNase A (Ambion), and analyzed using a linear ion trap/orbitrap hybrid mass spectrometer (LTQ Orbitrap XL, Thermo Fisher Scientific) including a custom-made nanospray ion source and a splitless nano HPLC system (DiNa, KYA Technologies) as described in the reference (Suzuki et al., 2007; Ohira, T. & Suzuki, T. Precursors of tRNAs are stabilized by methylguanosine cap structures. Nat Chem Biol 12, 648-655, doi: 10.1038/nchembio.2117 (2016)). When the Y site was analyzed, tRNA was treated with acrylonitrile, Y was cyanoethylated (Mengel-Jorgensen and Kirpekar, 2002), and then analyzed by RNA-MS. For the dephosphorylation experiment of RNA fragments with U^{p}47 shown in Extended Data FIG. 4a, b, RNase T₁ digestion was performed in the presence of 0.01 U/μL bacterial alkaline phosphatase (BAP C75; Takara Bio). In order to accurately identify the modification site on tRNA, each RNA fragment was degraded in the device by collision-induced dissociation (CID). The normalized collision energy of LTQ Orbitrap XL was set to 40%. Product ions appearing in the CID spectra were assigned using a Mongo Oligo Mass Calculator v2.08 (https://mods.rna.albany.edu/masspec/Mongo-Oligo).

When nucleoside analysis was performed, 800 ng (24 μmol) of the isolated and purified tRNA^{Val3} was digested in a 20 mM NH₄OAc (pH 5.2) solution containing 0.09 U nuclease P1 (FUJIFILM Wako Pure Chemical Corporation) at 50° C. for 1 hour, ⅛ volume of 1 M trimethylamine-HCl (TMA-HCl, pH 7.2) and 0.06 U phosphodiesterase I (Worthington Biochemical Corporation) were then added, and the mixture was incubated at 37° C. for 1 hour. After the reaction, 0.08U BAP was additionally added and the mixture was incubated at 50° C. for 1 hour. Then, 9 times the amount of acetonitrile was added, and LC-MS/MS analysis was performed with some modifications from the references (Sakaguchi, Y., Miyauchi, K., Kang, B. I. & Suzuki, T. Nucleoside Analysis by Hydrophilic Interaction Liquid Chromatography Coupled with Mass Spectrometry. Methods Enzymol 560, 19-28, doi: 10.1016/bs.mie.2015.03.015 (2015); Miyauchi, K., Kimura, S. & Suzuki, T. A cyclic form of N6-threonylcarbamoyladenosine as a widely distributed tRNA hypermodification. Nat Chem Biol 9, 105-111, doi: 10.1038/nchembio. 1137 (2013)). Isolation of the sample by LC was performed using a ZIC-CHILIC column (3 μm particle size, 2.1×150 mm; Merck) at a flow rate of 100 μL/min according to a multi-step linear concentration gradient of 5 mM ammonium acetate (pH 5.3) (solvent A) and acetonitrile (solvent B) (90-50% B for 30 minutes, 50% B for 10 minutes, 50-90% B for 5 minutes, initialized at 90% B). The isolated eluate was analyzed by a Q Exactive Hybrid Quadrupole-Orbitrap Mass Spectrometer (Thermo Fisher Scientific) as a direct ESI source.

When nucleotide analysis was performed, 800 ng (24 μmol) of tRNA fractions or individual tRNAs was digested in a 20 mM NH₄OAc (pH 5.2) solution containing 0.09 U nuclease P1 at 50° C. for 1 hour, 9 times the amount of acetonitrile was then added, and LC-MS analysis was performed. Isolation of the sample by LC was performed using a ZIC-CHILIC column according to a multi-step linear concentration gradient (90-50% B for 30 minutes, 50% B for 10 minutes, 50-90% B for 5 minutes, initialized at 90% B), and analysis was performed using a Q Exactive Hybrid Quadrupole-Orbitrap mass spectrometer (Thermo Fisher Scientific) or LTQ Orbitrap XL (Thermo Fisher Scientific).

The acquired LC-MS data was analyzed using Xcalibur 4.1 (Thermo Fisher Scientific) and then plotted using Canvas X (Nihon poladigital k.k).

5. Isolation, Purification and Detection of pN³²⁴p

Five A₂₆₀ units of S. tokodaii tRNA fractions were completely digested with nuclease P1, and the digest containing pN³²⁴m⁵C dinucleotides was then subjected to a periodic acid oxidation treatment with 10 mM NaIO₄. After the sample was incubated on ice in the dark place for 1 hour, 1 M L-rhamnose was added to stop the reaction, and the mixture was incubated for 30 minutes. Then, an equal volume of 2 M Lysine-HCl (pH 8.5) was added, the mixture was incubated at 45° C. for 90 minutes, and a β-elimination reaction was performed. In order to purify pN³²⁴p, anion exchange chromatography was performed using a Q Sepharose Fast Flow column (GE Healthcare) equilibrated with 20 mM triethylammonium bicarbonate (TEAB) (pH 8.2). The fractions eluted with 2 M TEAB were collected and then vacuum-dried. The pellets were dissolved in ultrapure water and mixed with an equal volume of chloroform, and the mixture as then centrifuged at 20,000 g, 4° C. for 5 minutes. The supernatant was collected and vacuum-dried again. This step was repeated 5 times. The obtained purified product was mixed with 9 times the amount of acetonitrile, and LC-MS/MS analysis was performed. LC-MS/MS was performed using an LCQ-Advantage ion trap mass spectrometer (Thermo Scientific) including an ESI source and an HP1100 LC system (Agilent Technologies). Isolation by LC was performed using a ZIC-HILIC column (3.5 μm; pore size, 100 A; internal diameter, 2.1×150 mm; Merck) at a flow rate of 100 μL/min according to a multi-step concentration gradient of 5 mM formic acid (pH 3.4) (solvent A) and acetonitrile (solvent B) (90-70% B for 25 minutes, 70-10% B for 15 minutes, 10% B for 5 minutes, initialized at 90% B).

### 6. Expression and Purification of Recombinant Proteins

Synthetic genes for the arkI genes derived from T. kodakarensis, Methanocaldococcus fervens, P. oguniense, A. aeolicus, Nautilia profundicola, and Leptolyngbya sp. PCC7376 were designed with codons optimized for expression in E. coli and synthesized by GENEWIZ or Thermo Fisher Scientific. Each gene was amplified by PCR using the synthesized genes as a template and specific primers (shown in Table 2-1 to 2-2) and cloned into a pE-SUMO-TEV vector by the SLICE method (Zhang, Y., Werling, U. & Edelmann, W. SLICE: a novel bacterial cell extract-based DNA cloning method. Nucleic Acids Res 40, 12 (2012)). The N. viennensis arkI gene was amplified by PCR using the genomic DNA as a template and specific primers (shown in Table 1), and inserted between the BamHI site and the NotI site of the pE-SUMO-TEV vector.

Table 1: Partial sequences of E. coli tRNA modified with NvArkI in cells

TABLE 1
Partial sequences of E. coli tRNA modified with
NvArkl in cells

| tRNA | Sequence (44:52) |
|---|---|
| Ala1A, Ala1B | AGGUCUGCGG (SEQ ID NO. 9) |
| Ala2 | AGGUCAGCGG (SEQ ID NO. 10) |
| Arg1, Arg2 | CGGUCGGAGG (SEQ ID NO. 11) |
| Asp | GGGUCGCGGG (SEQ ID NO. 12) |
| Gly3 | GGGUCGCGAG (SEQ ID NO. 13) |
| His | UUGUCGUGGG (SEQ ID NO. 14) |
| Ile1 | AGGUCGGUGG (SEQ ID NO. 15) |
| Ile2 | UGGUCGCUGG (SEQ ID NO. 16) |
| Lys | UGGUCGCAGG (SEQ ID NO. 17) |
| fMet1 | AGGUCGUCGG (SEQ ID NO. 18) |
| Met | GGGUCACAGG (SEQ ID NO. 19) |
| Pro1, Pro2, Pro3 | GGGUCGGAGG (SEQ ID NO. 20) |
| Thr2 | AGGUCGUAGG (SEQ ID NO. 21) |
| Thr3 | AGGUCGGCAG (SEQ ID NO. 22) |
| Val1 | GGGUCGGCGG (SEQ ID NO. 23) |
| Val2A | GGGUCGGUGG (SEQ ID NO. 24) |
| Val2B | GGGUCGUUGG (SEQ ID NO. 25) |

In Table 1, the primary sequence was extracted from position 44 (V-loop) to position 52 (T-stem). The RNA fragments generated by RNase T1 are shown in bold.

E. coli BL21 (DE3) or Rosetta2 (DE3) transformed with each arkI expression vector was cultured in a 250 mL or 1 L LB medium containing 50 μg/mL kanamycin (Kan) and 20 μg/mL chloramphenicol (Cam) as necessary. The expression was induced with 0.1 mM or 1 mM IPTG, or 2% (wt/vol) lactose at 37° C. for 3-4 hours when OD₆₁₀ reached 0.4-0.6. The expression of P. oguniense ArkI was induced at 18° C. overnight. The collected cultured cells were re-suspended in a lysis buffer (50 mM HEPES-KOH (pH 8.0), 150 mM KCl, 2 mM MgCl₂, 20 mM imidazole, 12% (v/v) Glycerol, 1 mM 2-mercaptoethanol, and 1 mM PMSF), and disrupted by ultrasonication, and centrifugation was then performed at 15,000 g, 4° C. for 15 minutes. For recombinant ArkI proteins derived from T. kodakarensis, M. fervens, P. oguniense, A. aeolicus, the collected supernatant was incubated at 60° C. for 20 minutes, and centrifuged at 15,000 g, 4° C. for 15 minutes. The recombinant proteins were purified by affinity chromatography using a Ni-Sepharose 6 Fast Flow column (GE Healthcare) and eluted with a lysis buffer containing 300 mM imidazole, and then purified by gel filtration using a PD-10 column (GE Healthcare) to remove imidazole. The recombinant ArkI proteins (NvArkI) derived from N. viennensis were eluted using a HisTrap column (GE Healthcare) according to a linear concentration gradient of 0-500 mM imidazole, and imidazole was then removed by dialysis using Slide-A-Lyzer™ Dialysis Cassette (Thermo Fisher Scientific). The purified recombinant proteins were digested with Ulp1 at 4° C. overnight to cut off the His₆-SUMO tag, and then passed through a Ni-Sepharose 6 Fast Flow column in order to remove the tag moiety. Recombinant ArkI proteins derived from M. fervens (MfArkI) and Leptolyngbya sp. PCC7376 (LeArkI) were used in tRNA phosphorylation assays without cutting off the His₆-SUMO tag because they were found to aggregate when the tag was cut off. The concentration of each purified protein was quantified by the Bradford method using bovine serum albumin as a standard.

In order to prepare a large amount of T. kodakarensis ArkI required for X-ray crystal structure analysis, the E. coli BL21 (DE3) strain transformed with a pE-SUMO-TkArkI vector was cultured in a 2 L LB medium supplemented with 50 μg/mL Kan, and when OD₆₁₀ reached 0.4, expression was induced with 0.1 mM IPTG at 25° C. overnight. The cultured cells were collected and suspended in a lysis buffer (50 mM HEPES-KOH (pH 8.0), 150 mM KCl, 2 mM MgCl₂, 20 mM imidazole, 12% (v/v) Glycerol, 1 mM 2-mercaptoethanol, and 1 mM PMSF) and then disrupted by ultrasonication. The recombinant proteins were purified using a HisTrap column according to a linear concentration gradient of 20-520 mM imidazole. Fractions containing TkArkI were collected and digested with Ulp1 at 4° C. overnight to cut off the tag, then passed through a Ni-Sepharose 6 Fast Flow column to remove the tag fragment. Then, the resulting passed fractions were filtered through a 0.45 UM PVDF membrane to remove the remaining resin. In order to purify TkArkI from the passed fractions, affinity chromatography was performed using a HiTrap Heparin HP column (GE Healthcare), and elution was performed according to a linear concentration gradient of 150-1,150 mM KCl. In addition, size-exclusion chromatography was performed using a Superdex75 10/300 GL column (GE Healthcare) equilibrated with a buffer containing 20 mM Tris-HCl (pH 8.0), 150 mM NaCl and 10 mM 2-mercaptoethanol. The purified TkArkI was concentrated to 5.74 mg/mL and then stored at −80° C.

In order to obtain the kptA gene derived from T. kodakarensis, amplification was performed by PCR using the genomic DNA of T. kodakarensis as a template and primers shown in Table 1, and the gene was then cloned to a pE-SUMO-TEV vector, and thereby pE-SUMO-TEV-tkkptA was obtained. The E. coli Rosetta2 (DE3) strain transformed with this vector was cultured in a 1 L LB medium containing 50 μg/mL Kan and 20 μg/mL Cam, and when OD₆₁₀ reached 0.6, expression was induced with 0.1 mM IPTG at 37° C. for 3 hours. TkKptA was purified as described above. The Tpt1p gene was amplified by PCR using the genomic DNA of Saccharomyces cerevisiae BY4742 strain as a template and primers shown in Table 1, and inserted between the NdeI site and the XhoI site of the pET21b (Merck) vector. Tpt1p was purified as described above.

### 7. Removal of 2′-Phosphate Group of U^{p}47 by Tpt1p

The 2′-phosphate group of U^{p}47 was removed by Tpt1p according to the reference (Culver, G. M., McCraith, S. M., Consaul, S. A., Stanford, D. R. & Phizicky, E. M. A 2′-phosphotransferase implicated in tRNA splicing is essential in Saccharomyces cerevisiae. J Biol Chem 272, 13203-13210 (1997)). The isolated and purified tRNA or tRNA fraction was incubated in a 25 μL reaction solution containing 20 mM Tris-HCl (pH 7.4), 0.5 mM EDTA (pH 8.0), 1 mM NAD⁺, 2.5 mM spermidine, 0.1 mM DTT, 0.9 UM tRNA, and 0.1 μg/μL Tpt1p at 30° C. for 3 hours. After the reaction, tRNA was extracted according to a treatment with phenol/chloroform, collected by precipitation with ethanol, and purified using a Centri-Sep spin column (Princeton Separations). When tRNA from which the 2′-phosphate group for X-ray crystal structure analysis was removed was prepared, S. tokodaii tRNA^{Val3} (202.5 μg) was dephosphorylated with Tpt1p in a 200 μL reaction solution.

8. Measurement of Thermostability of tRNA

S. tokodaii tRNA^{Val3} (25 μmol) with or without U^{p}47 was dissolved in a degassed buffer containing 50 mM Tris-HCl (pH 7.4), 100 mM NaCl, and 1 mM MgCl₂, incubated at 80° C. for 5 minutes, cooled to 25° C. at a rate of 0.1° C./sec, and then put into an 8-series multi-micro UV quartz cell (pass length: 10 mm). The hyperchromic effect of tRNA was monitored using a UV-Vis spectrophotometer (V-630; JASCO). The temperature gradient was set as follows: 25° C. for 30 sec, 25-40° C. at 5° C./min, 40° C. for 5 minutes, and 40-105° C. at 0.5° C./min. The melting temperature was calculated by Spectra Manager v2 (JASCO). The melting curve was created using Microsoft Excel (Microsoft Corporation).

9. RNase Probing of tRNA

The 3′ end of S. tokodaii tRNA^{Val3} (25 μmol) with or without U^{p}47 was labeled with ³²P by linking cytidine 3′,5′-bisphosphate [5′-³²P] (PerkinElmer). The labeled tRNA was isolated with a 7.5% (wt/vol) polyacrylamide gel containing 7 M urea, 1×TBE, and 10% (v/v) Glycerol, and purified by gel extraction. Then, the purified ³²P-labeled tRNA and the carrier S. tokodaii tRNA fraction were mixed to 100,000 count per minute (c.p.m.)/A₂₆₀ unit and precipitated with ethanol. The obtained pellets were dissolved in ultrapure water to a concentration of 0.1 A₂₆₀ units/μL. In order to perform RNase degradation, the ³²P-labeled tRNA (0.1 A₂₆₀ units, 10,000 c.p.m.) was incubated at 65° C. in a reaction solution containing 10 mM HEPES-KOH (pH 7.6), 0.5 mM MgCl₂, 100 mM NaCl, and 0.1 unit/μL RNase I (Promega). After the reaction started, aliquots were taken from the reaction solution at 1, 3, 5, 10, 15, and 30 minutes, mixed thoroughly with chilled phenol/chloroform/isoamyl alcohol (25:24:1, pH 7.9) to stop the reaction, and then centrifuged at 15,000 g, 4° C., for 15 minutes. The supernatant was collected, treated with an equal volume of chloroform, and then centrifuged at 15,000 g, 4° C., for 5 minutes. The supernatant was collected and mixed with a 2×loading solution (2×TBE, 7 M urea, 13.33% (wt/vol) sucrose, 0.05% (wt/vol) XC, and 0.05% (wt/vol) BPB) and subjected to 10% denaturing PAGE. The gel was exposed to an imaging plate, and ³²P-labeled tRNA was visualized using an FLA-7000 imaging analyzer (Fujifilm). The graph was created using Microsoft Excel (Microsoft Corporation).

10. Crystallization of S. tokodaii tRNA^{Val3}

The isolated and purified S. tokodaii tRNA^{Val3} (500 μg) was incubated in an annealing buffer (50 mM HEPES-KOH (pH 7.6), 5 mM MgCl₂, and 1 mM DTT) solution at 80° C. for 5 minutes, and cooled to 25° C. at a rate of 0.1° C./sec for refolding. Then, in order to purify tRNA^{Val3}, elution was performed using a Mono Q 5/50 GL column (GE Healthcare) according to a linear concentration gradient of 200-1,000 mM NaCl. The main peak of tRNA^{Val3} was collected, precipitated with isopropanol for collection, then dissolved again in ultrapure water, and precipitated with ethanol. Tpt1p-treated tRNA^{Val3} was also prepared by the same procedure as above. The purified tRNA was dissolved in a buffer containing 10 mM Tris-HCl (pH 7.1) and 5 mM MgCl₂ to a concentration of 50 μM. 1 μL of the tRNA solution was mixed with 1 μL Natrix 2 No. 32 (80 mM NaCl, 12 mM spermine-4HCl, 40 mM NaCacodylate-3H₂O (pH 7.0), and 30% (v/v) MPD) (Hampton Research) on a silicon-coated glass, and tRNA was crystallized by the hanging drop vapor diffusion method at 20° C.

11. Crystallization of T. kodakarensis ArkI

Before crystallization, the concentration of TkArkI was adjusted to 5 mg/mL. 1 μL of a protein solution was mixed with 0.5 μL of a reservoir solution containing 25% (v/v) ethylene glycol, and TkArkI was crystallized by the hanging drop vapor diffusion method at 20° C.

### 12. Data Collection and Determination of Crystal Structure

The crystal structure data was collected at the beamline BL17A of the synchrotron radiation facility in the High Energy Accelerator Research Organization (KEK), Japan. For data collection of tRNA^{Val3} crystals, the crystals were prevented from freezing with the reservoir solution. For data collection of native TkArkI crystals, the crystals were prevented from freezing with a solution containing 25% (v/v) ethylene glycol, 2 mM MgCl₂, and 1 mM ATP. For data collection of iodinated derivatives of TkArkI, the crystals were immersed for a short time in a solution containing 300 mM potassium iodide and 22.5% (v/v) ethylene glycol, and were prevented from freezing, and a diffraction data set was collected at a wavelength of 1.5 A. The obtained data set was indexed, integrated, and scaled using xds (Kabsch, 2010). The initial phase of tRNA^{Val3} was determined by molecular substitution using Phaser (McCoy, A. J. et al. Phaser crystallographic software. J Appl Crystallogr 40, 658-674, doi: 10. 1107/s0021889807021206 (2007)). The model was constructed using the structure of Thermus thermophilus tRNA^{Val} (PDB ID: 1IVS) (Fukai, S. et al. Mechanism of molecular interactions for tRNA (Val) recognition by valyl-tRNA synthetase. RNA 9, 100-111, doi: 10.1261/rna.2760703 (2003)). The initial phase of TkArkI was determined by a single-wavelength anomalous diffraction method (SAD method) using the anomalous diffraction effect of iodine. The position of iodine was determined using SHELX (Sheldrick, 2008), and the initial phase was calculated using Phaser. Density correction and initial model construction were performed using RESOLVE (Terwilliger, 2000). In addition, modification was performed by Coot (Emsley, P., Lohkamp, B., Scott, W. G. & Cowtan, K. Features and development of Coot. Acta Crystallogr D Biol Crystallogr 66, 486-501, doi: 10.1107/s0907444910007493 (2010)), and refinement was performed by Phenix (Adams, P. D. et al. PHENIX: a comprehensive Python-based system for macromolecular structure solution. Acta Crystallogr D Biol Crystallogr 66, 213-221, doi: 10. 1107/s0907444909052925 (2010)). The crystal structure and electron density map were drawn using Pymol, Cuemol, or Coot. The torsion angle of tRNA was analyzed using DSSR software (Lu, X. J., Bussemaker, H. J. & Olson, W. K. DSSR: an integrated software tool for dissecting the spatial structure of RNA. Nucleic Acids Res 43, e142, doi: 10.1093/nar/gkv716 (2015)).

The X-ray crystal structures obtained from this study were deposited under accession codes 7VNV, 7VNW, and 7VNX in Protein Data Bank.

### 13. Analysis of Ligand Bound to TkArkI

TkArkI (100 μmol) affinity-purified using a HiTrap Heparin HP column (GE Healthcare) was mixed with tracers [¹⁵N] adenosine (10 μmol) and [¹⁵N] guanosine (10 μmol) and 4 times the amount of methanol, an equal volume of chloroform, and 3 times the amount of water were then added, and mixed vigorously. The mixture was centrifuged at 15,000 g, 4° C. for 1 minute, and denatured proteins were removed. The supernatant was collected, vacuum-dried, and then redissolved in 20 μL of ultrapure water. Half of the obtained extract was subjected to LC-MS analysis. The tracers were prepared by dephosphorylating [¹⁵N] ATP and [¹⁵N] GTP as follows: 1,000 pmol of [¹⁵N] ATP (Silantes) and [¹⁵N] GTP (Silantes) were treated in a 20 mM NH₄OAc (pH 8.0) solution containing 0.04 U Alkaline Phosphatase (PAP, derived from Shewanella sp. SIB1, BioDynamics Laboratory) at 60° C. for 30 minutes. After the dephosphorylation reaction, PAP was thermally denatured at 95° C. for 5 minutes.

14. Construction of Gene-Deficient Strains of T. kodakarensis

The gene-deficient strain of T. kodakarensis was constructed by pop-in/pop-out recombination according to the reference (Sato et al., 2005). The 5′-side and 3′-side flanking regions (about 1,000 bp) of arkI and kptA of T. kodakarensis were amplified by PCR using a specific primer set (shown in Table 1) and the genomic DNA as a template, and the genes were inserted into a pUD3 vector with a pyrF marker (Kobori, H. et al. Characterization of NADH oxidase/NADPH polysulfide oxidoreductase and its unexpected participation in oxygen sensitivity in an anaerobic hyperthermophilic archaeon. J Bacteriol 192, 5192-5202, doi: 10.1128/JB.00235-10 (2010)) to obtain pUD3-arkI and pUD3-kptA. T. kodakarensis KU216 strain (ΔpyrF) was transformed with pUD3-arkI or pUD3-kptA, and uracil-non-requiring transformants generated by pop-in recombination were selected using an uracil-free ASW-AA-S⁰ medium plate. Next, the selected transformants were seeded on an ASW-AA-S⁰ medium plate to which 5-FOA was added to obtain 5-FOA-resistant transformants obtained by pop-out recombination. Among these, the arkI or kptA gene-deficient strain was selected by genomic PCR using a specific primer set (shown in Table 1). The arkI and queE double gene-deficient strain (ΔarkI/queE::Tn) was constructed by deleting the arkI gene from FFH05 (queE::Tn), which was isolated from a random mutation library of T. kodakarensis (Orita, I. et al. Random mutagenesis of a hyperthermophilic archaeon identified tRNA modifications associated with cellular hyperthermotolerance. Nucleic Acids Res, doi: 10.1093/nar/gky1313 (2019)). The probes and primers for the T. kodakarensis strain used in this study are listed in Tables 2-1 to 2-3.

TABLE 2-1

| | | |
|---|---|---|
| Experiment | Sequence (5′-3′) | No |

tRNA isolation

| | | |
|---|---|---|
| tRNAVal3 | 5′ ECamino-AGGGCGGCGTCCTAACCAGGCTAGACGACGGGCCC-3′ | 26 |
| tRNALys2 | 5′ ECamino-GTTAAAAGCCCGCCGCTCTACCTGGCTGAGCTACGGGCCC-3′ | 27 |
| tRNAThr3 | 5′ ECamino-TTACAAGGCCGGCGCTCTAACCAGGCTGAGCTACAGCGGC-3′ | 28 |
| tRNAGly3 | 5′ ECamino-GGCCAGCGTCCTAATCCAGACTAGACGACGGCCGC-3′ | 29 |
| tRNAArg4 | 5′ ECamino-TCCGAAGGCCGGCGCTCTATCCTGGCTGAGCTACGGGTCC-3′ | 30 |
| tRNAPro2 | 5′ ECamino-GGCCTGCATCCTAGTCCAGGCTAGACGACGGTCCC-3′ | 31 |
| tRNACys | 5′ ECamino-TGCGGCCCGCCGCCTTAACCTCTCGGCCACCCCGGC-3′ | 32 |
| tRNAGln2 | 5′ ECamino-GGCCAAAGCCCTCGATCCTTGACCACTAGACGACCCGGCT-3′ | 33 |
| tRNALeu4 | 5′ ECamino-CCTAAGCCCGACCCCTTTGACCTTGCTCGGGCACCCCCGC-3′ | 34 |
| tRNASer3 | 5′ ECamino-CTCTCAAGGCCAGCCCCTTAGTCCACTCGGGCACCCCGGC-3′ | 35 |
| tRNAIle2 | 5′ ECamino-GAGCCGGGCGCTCTACCGGGCTAAGCTACGGGCCC-3′ | 36 |
| tRNAPhe | 5′ ECamino-GTCTTCAGCCGGGCGCTCTCCCGGGCTGAGCTACG-3′ | 37 |

Gene cloning

| | | |
|---|---|---|
| ScTpt1_Fw | 5′-GACTGACTCATATGCGCCAGGTACTACAAAA-3′ | 38 |
| ScTpt1_Rv | 5′-GACTCTCGAGTATCTTTTCGAGCGGTATGTTTC-3′ | 39 |
| TkArkl_Fw | 5′-AAACTTGTACTTCCAAGGAATGACCTTCGAGCATATC-3′ | 40 |
| TkArkl_Rv | 5′-ATTCGGATCCCATATGGGATCACAGACCGCTCAGTAAAG-3′ | 41 |
| MfArkl_Fw | 5′-AAACTTGTACTTCCAAGGAATGGCCATTAAAAAAGAAATTC-3′ | 42 |
| MfArkl_Rv | 5′-ATTCGGATCCCATATGGGATCACAGTTTTTTATAGGTTTTGGC-3′ | 43 |
| NvArkl_Fw | 5′-GTGATCTGGGATCCATGTCTCAACAGAGAGCC-3′ | 44 |
| NvArkl_Rv | 5′-GTGATCTGGCGGCCGCTCAGGCAACAACAACAAG-3′ | 45 |
| AaArkl_Fw | 5′-AAACTTGTACTTCCAAGGAATGAAATTTAGCGAGTTCATTA-3′ | 46 |
| AaArkl_Rv | 5′-ATTCGGATCCCATATGGGATCAGCTCAGCACTTTGCG-3′ | 47 |
| NpArkl_Fw | 5′-CCGCGAACAGATTGGAGGTATGATCATCTTCGAGAAAAAGGG-3′ | 48 |
| NpArkl_Rv | 5′-CTTGGAAGTACAAGTTTTCTCAGCTTTCATCAATCAGGCGAATA-3′ | 49 |
| LeArkl_Fw | 5′-CCGCGAACAGATTGGAGGTATGGCAGGTAGCCAGTTTC-3′ | 50 |
| LeArkl_Rv | 5′-CTTGGAAGTACAAGTTTTCTCACAGATTCAGGCTGCTCAG-3′ | 51 |
| TkKptA_Fw | 5′-AAACTTGTACTTCCAAGGAATGAAGCCAGAGCGGAAG-3′ | 52 |
| TkKptA_Rv | 5′-ATTCGGATCCCATATGGGACTAAACCGCCAGGGTTATACA-3′ | 53 |
| pSUMO_Fw | 5′-TCCCATATGGGATCCG-3′ | 54 |

TABLE 2-2

| | | |
|---|---|---|
| pSUMO_Rv | 5′-TCCTTGGAAGTACAAGTTT-3′ | 55 |
| pSUMO_Fw2 | 5′-GAAAACTTGTACTTCCAAGGAT-3′ | 56 |
| pSUMO_Rv2 | 5′-ACCTCCAATCTGTTCGCG-3′ | 57 |

Gene knockout

| | | |
|---|---|---|
| TkArkl_Upstream_pUD3_Fw | 5′-GCTTGCATGCCTGCAGTCCTTCCATAGGTAGATG-3′ | 58 |
| TKArkl_Upstream_Rv | 5′-ACTATTTAAGCTTATCGCGTCAAGAGAACCGGAAGCCTC-3′ | 59 |
| TkArkl_Downstream_Fw | 5′-ACGCGATAAGCTTAAATAGT-3′ | 60 |
| TkArkl_Downstream_pUD3_Rv | 5′-CCTCTAGAGTCGACCAGTCCAAACCCTCGATATAA-3′ | 61 |
| TkKptA_Upstream_pUD3_Fw | 5′-CCAAGCTTGCATGCCTGCAACCATCCGCAAAGGGGAG-3′ | 62 |
| TkKptA_Upstream_Rv | 5′-GAAACTCGCATGGGCTGACGCTAAAAACCTCACGGGA-3′ | 63 |
| TkKptA_Downstream_Fw | 5′-GTCAGCCCATGCGAGTTTC-3′ | 64 |
| TkKptA_Downstream_pUD3_Rv | 5′-GGATCCTCTAGAGTCGACCCTTTGCTATCAAAAGCCGC-3′ | 65 |
| pUD3_Fw | 5′-GGTCGACTCTAGAGGATCCC-3′ | 66 |
| pUD3_Rv | 5′-TGCAGGCATGCAAGCTTG-3′ | 67 |

in vitro T7 transcription

| | | |
|---|---|---|
| Tk tRNAVal3_Fw | 5′-CAGTAATACGACTCACTATAGGGCCCGTGGTCTAGATGGTT-3′ | 68 |
| Tk tRNAVal3_Body | 5′-CCGTGGTCTAGATGGTTATGACGCCACCCTTACAAGGTGGAGGTCCGG-3′ | 69 |
| Tk tRNAVal3_Rv | 5′-TGGTGGGCCCGCGGGGATTCGAACCCCGGACCTCCACCTTG-3′ | 70 |
| Tk tRNAVal3_G5:C68_Fw | 5′-CAGTAATACGACTCACTATAGGGCGCGTGGTCTAGATG-3′ | 71 |
| Tk tRNAVal3_G5:C68_Body | 5′- | 72 |
| | AGGGCGCGTGGTCTAGATGGTTATGACGCCACCCTTACAAGGTGGAGGTCCGGGGTT-3′ | |
| Tk tRNAVal3_G5:C68_Rv | 5′-TGGTGGGCGCGCGGGGATTCGAACCCCGGACCTCCACCTT-3′ | 73 |

Mutation study

| | | |
|---|---|---|
| TkArkl_K32A_Fw | 5′-GCTGGCAGCGGGTACCACCTCTTTAGTG -3′ | 74 |
| TkArkl_K32A_Rv | 5′-GTACCCGCTGCCAGCGGTTCCACACCAC -3′ | 75 |
| TkArkl_G33A_Fw | 5′-GCAAAGGCGACCACCTCTTTAGTGTTTAC-3′ | 76 |
| TkArkl_G33A_Rv | 5′-GGTGGTCGCCTTTGCCAGCGGTTCCAC-3′ | 77 |
| TkArkl_K51A_Fw | 5′-GGTGATTGCGCTGCAGCGCCCGGACAGC-3′ | 78 |
| TkArkl_K51A_Rv | 5′-GCTGCAGCGCAATCACCACTTTGCGACC-3′ | 79 |
| TkArkl_E65A_Fw | 5′-GAAAAAGCAGCTGAGCTGACCAAAATC-3′ | 80 |
| TkArkl_E65A_Rv | 5′-CTCAGCTGCTTTTTCAAAATTGCTACG -3′ | 81 |
| TkArkl_R95A_Fw | 5′-CTGATTGCGGAATTTGCCGAGGGCGAAC-3′ | 82 |
| TkArkl_R95A_Rv | 5′-CAAATTCCGCAATCAGATACGGTAAACC-3′ | 83 |
| TkArkl_H130A_Fw | 5′-ATTGATGCTGGCCAGATCCAAGGTGGC-3′ | 84 |
| TKArkl_H130A_Rv | 5′-CTGGCCAGCATCAATGCCCAGACGATC-3′ | 85 |

TABLE 2-3

| | | |
|---|---|---|
| TkArkl_Q132A_Fw | 5′-CATGGCGCGATCCAAGGTGGCAAACAC-3′ | 86 |
| TkArkl_Q132A_Rv | 5′-TTGGATCGCGCCATGATCAATGCCCAG-3′ | 87 |
| TkArkl_K137A_Fw | 5′-GGTGGCGCGCACATTATCATCGGCGAG-3′ | 88 |
| TkArkl_K137A_Rv | 5′-AATGTGCGCGCCACCTTGGATCTGGCC-3′ | 89 |
| TkArkl_D149A_Fw | 5′-CTGATCGCGTTTGAGAAGGCCGGCTTC-3′ | 90 |
| TkArkl_D149A_Rv | 5′-CTCAAACGCGATCAGATACACATCCTC-3′ | 91 |
| TkArkl_N160A_Fw | 5′-CCGAACGCGCTGACCAGCGCCATGGCC-3′ | 92 |
| TkArkl_N160A_Rv | 5′-GGTCAGCGCGTTCGGTTTGCGGAAGCC-3′ | 93 |
| TkArkl_T162A_Fw | 5′-AATCTGGCGAGCGCCATGGCCATGATCT-3′ | 94 |
| TkArkl_T162A_Rv | 5′-GGCGCTCGCCAGATTGTTCGGTTTGCG-3′ | 95 |
| TkArkl_Y200A_Fw | 5′-CGCCACGCGAAACGCACCGGTAGTCTG-3′ | 96 |
| TkArkl_Y200A_Rv | 5′-GCGTTTCGCGTGGCGTAAAGCATCTTTC-3′ | 97 |
| TkArkl_K201A_Fw | 5′-CACTACGCGCGCACCGGTAGTCTGAGT -3′ | 98 |
| TkArkl_K201A_Rv | 5′-GGTGCGCGCGTAGTGGCGTAAAGCATC-3′ | 99 |
| TkArkl_R202A_Fw | 5′-ACTACAAAGCGACCGGTAGTCTGAGTCGT-3′ | 100 |
| TkArkl_R202A_Rv | 5′-ACCGGTCGCTTTGTAGTGGCGTAAAGC-3′ | 101 |

### 15. Analysis of Growth Phenotype

T. kodakarensis KU216 (WT), FFH05 (queE::Tn), ΔarkI, and ΔarkI/queE::Tn strains pre-cultured overnight in MA-YT-Pyr mediums at 83° C. were subcultured in 8 mL of fresh MA-YT-Pyr mediums to an OD₆₀₀ of 0.01, and cultured at 83° C., 87° C., and 91° C. Growth was monitored by measuring OD₆₀₀ every 2 hours using a WPA S1200 diode array spectrophotometer (Biochrom). The graph was created using Microsoft Excel (Microsoft Corporation).

16. In Vitro Transcription Synthesis of tRNA

For in vitro transcription synthesis of T. kodakarensis tRNA^{Val3} and its G5-C68 variant using T7 RNA polymerase (Sampson and Uhlenbeck, 1988), a template DNA was prepared by PCR using a synthetic oligo DNA (shown in Table 1). The transcription synthesis reaction was performed in a reaction solution containing 40 mM Tris-HCl (pH 7.5), 24 mM MgCl₂, 5 mM DTT, 2.5 mM spermidine, 0.01% (v/v) Triton X-100, 0.005% (wt/vol) BSA, 0.8 μg/mL T7 RNA polymerase, 1 μg/mL pyrophosphatase, 30 nM template DNA, 2 mM ATP, 2 mM CTP, 2 mM UTP, 2 mM GTP, and 10 mM GMP at 37° C. overnight. After the reaction, the in vitro transcription synthesis product was purified by extraction with a treatment of phenol/chloroform and desalting with a PD-10 column (GE Healthcare). The obtained in vitro transcription synthesis product was isolated by 10% denaturing PAGE and then stained with toluidine blue. The stained band was excised from the gel and eluted from the gel with an elution buffer, gel pieces were then removed by filtration through a filter, and the in vitro transcription synthesis product was collected by precipitation with ethanol.

17. Phosphorylation of tRNA by ArkI In Vitro

The U^{p}47 formation reaction using TkArkI was performed in a reaction solution containing 50 mM HEPES-KOH (pH 7.5), 1 mM MgCl₂, 1 mM MnCl₂, 1 mM DTT, 10% (v/v) Glycerol, 0.5 mM ATP, 0.9 UM tRNA fraction (derived from T. kodakarensis ΔarkI strain), and 1 μM TkArkI at 70° C. for 20 minutes. After the reaction, tRNA was extracted with acidic phenol/chloroform, desalted with an NAP-5 column (GE Healthcare), and collected by precipitation with isopropanol. When RNA-MS was performed, a solution containing the prepared tRNA was spotted on a nitrocellulose membrane (0.025 μm VSWP, MF-Millipore; Merck), and dialysis was performed against ultrapure water for 2 hours (drop dialysis). In an experiment to examine whether GTP can act as a phosphate group donor, 0.5 mM ATP or GTP was added to the reaction solution, the U^{p}47 formation reaction was performed using 0.5 UM TkArkI for 5 minutes, and RNA-MS analysis was then performed. The activity of each TkArkI variant was measured and confirmed by the transfer of the γ-phosphate group from [γ-³²P] ATP to tRNA in the same method as in the reaction kinetics analysis of TkArkI (see below). The phosphorylation reaction of tRNA was performed in 8 μL of the reaction solution at 70° C. for 15 minutes. In order to perform confirmation by PAGE, 4 μL of the reaction solution was mixed with 4 μL of a 2×loading solution, isolation by 10% denaturing PAGE was performed, the gel was then exposed to an imaging plate, and ³²P-labeled tRNA was visualized using an FLA-9000 (Fujifilm). Gel images were analyzed using Multi Gauge (Fujifilm). Bar graphs were created using R (R Foundation). The phosphorylation experiment using Total RNA was performed in 8 μL of a reaction solution containing 50 mM HEPES-NaOH (pH 7.5), 1 mM MgCl₂, 1 mM MnCl₂, 1 mM DTT, 10% (v/v) Glycerol, 100 UM [γ-³²P] ATP (3,000 mCi/mmol; Perkin Elmer), 1.8 μM TkArkI, and 50 ng/μL total RNA fraction (from T. kodakarensis ΔarkI strain) at 70° C. for 30 minutes. Then, 0.5 μL of 50 mM EDTA-NaOH (pH 8.0) was added, 4 μL of the reaction solution was mixed with 4 μL of a 2×loading solution, isolation by 10% denaturing PAGE was performed, and visualization was then performed in the same manner as above.

The U^{p}47 formation reaction experiment using other ArkI homologues was performed in a reaction solution (30 μL) containing 50 mM PIPES-NaOH (pH 6.9), 125 mM NaCl, 1 mM MgCl₂, 1 mM MnCl₂, 1 mM DTT, 10% (v/v) Glycerol, 500 UM ATP, 0.05 mg/mL BSA (Takara Bio), a 1 μM tRNA transcription synthesis product, and 0.5 UM ArkI proteins at 70° C. for 30 minutes. The reaction was performed at 45° C. for NvArkI. The reaction was performed at 50° C. for 60 minutes for NpArkI. After the reaction, tRNA was prepared as described above. In order to perform confirmation by PAGE, the U^{p}47 formation reaction was performed in a reaction solution (8 μL) containing 50 mM PIPES-NaOH (pH 6.9), 125 mM NaCl, 1 mM MgCl₂, 1 mM MnCl₂, 1 mM DTT, 10% (v/v) Glycerol, 100 μM [γ-³²P] ATP (3,000 mCi/mmol; Perkin Elmer), 0.1 mg/mL BSA, 0.75 UM recombinant ArkI homologue (NpArkI, NvArk I, and LeArkI), and 50 ng/μL of E. coli Total RNA. After the reaction, the reaction solution was mixed with a 2×loading solution, isolation by 10% denaturing PAGE was performed, and visualization was then performed in the same manner as above.

18. Dephosphorylation of tRNA Using TkKptA In Vitro

The U^{p}47 dephosphorylation reaction using TkKptA was performed in a reaction solution (30 μL) containing 20 mM Tris-HCl (pH 7.4), 0.5 mM EDTA (pH 8.0), 1 mM NAD⁺, 2.5 mM spermidine, 0.1 mM DTT, 0.9 UM T. kodakarensis tRNA fraction, and 0.1 μg/μL recombinant TkKptA at 60° C. for 1 hour. After the reaction, tRNA was extracted with acidic phenol/chloroform, desalted with an NAP-5 column (GE Healthcare), and then collected by precipitation with isopropanol. When RNA-MS analysis was performed, the prepared tRNA was desalted by drop dialysis in the same manner as above.

### 19. Reaction Kinetics Analysis of TkArkI and TkKptA

The U^{p}47 formation reaction using TkArkI was quantitatively evaluated by measuring the transfer of the γ-phosphate group from [γ-³²P] ATP to tRNA. In order to analyze reaction kinetics for the tRNA substrate, the phosphorylation reaction of tRNA was performed in a reaction solution (25 μL) containing 50 mM PIPES-NaOH (pH 6.9), 125 mM NaCl, 1 mM MgCl₂, 1 mM MnCl₂, 1 mM DTT, 10% (v/v) Glycerol, 100 UM [γ-³²P] ATP (1,500 mCi/mmol; Perkin Elmer), 0.05 mg/mL BSA, 0.05 UM TkArkI, and 0.1-5.0 UM in vitro transcribed T. kodakarensis tRNA^{Val3} at 70° C. For analyzing reaction kinetics for the ATP substrate, the ATP concentration was changed from 15.6 to 1,000 UM [γ-³²P] ATP (750 mCi/mmol; Perkin Elmer), and the tRNA concentration was increased to 1.0 μM. At each time point (2 minutes and 5 minutes), 8 μL of each solution was taken and mixed with an equal volume of a 2×loading solution (7 M urea, 0.2% (wt/vol) bromophenol blue, 0.2% (wt/vol) xylene cyanol, and 50 mM EDTA (pH 8.0)) to stop the reaction. After each sample was isolated by 10% denaturing PAGE, the gel was exposed to an imaging plate, and ³²P-labeled tRNA was measured by an FLA-9000 imaging analyzer. The kinetic parameter was calculated by Prism 7 (GraphPad).

The U^{p}47 dephosphorylation reaction using TkKptA was quantitatively evaluated by measuring a decrease in radioactivity of ³²P-labeled tRNA. The in vitro transcribed T. kodakarensis tRNA^{Val3} was phosphorylated with [γ-³²P] ATP using TkArkI as described above, and purified by gel extraction and precipitation with isopropanol. In addition, the same tRNA was phosphorylated with unlabeled ATP using TkArkI. ³²P-labeled tRNA and unlabeled tRNA were mixed, and the specific activity of ³²P-labeled tRNA was adjusted to 6,250 c.p.m./pmol in a buffer containing 50 mM HEPES-KOH (pH 7.6), 5 mM MgCl₂, and 1 mM DTT. Then, this ³²P-labeled RNA solution was incubated at 80° C. for 5 minutes, then cooled to room temperature, then precipitated with isopropanol, and then dissolved in ultrapure water to a concentration of 8 UM (50,000 c.p.m./μL). The dephosphorylation reaction of ³²P-labeled tRNA with TkKptA was performed in a reaction solution (30 μL) containing 50 mM PIPES-NaOH (pH 6. 9), 125 mM NaCl, 1 mM MgCl₂, 1 mM MnCl₂, 1 mM DTT, 10% (v/v) Glycerol, 1 mM NAD⁺, 0.05 mg/mL BSA, 1 nM TkKptA, and 12.5-800 nM ³²P-labeled tRNA at 70° C. At each time point (2 minutes and 5 minutes), 8 μL of each solution was taken and spotted on a Whatman 3 MM filter paper, and rapidly immersed in a 5% (wt/vol) trichloroacetic acid solution. The filter paper was washed three times for 15 minutes with an ice-cold 5% (wt/vol) trichloroacetic acid solution, then rinsed with ice-cold ethanol for 5 minutes, and dried in air. Radioactivity on the filter paper was measured by a liquid scintillation counter (Tri-Carb 2910TR; PerkinElmer). The kinetic parameter was calculated by Prism 7.

### 20. Dephosphorylation of U^{p}47 Using KptA In Vivo

The N. viennensis arkI gene was amplified by PCR, inserted into a pMW118 vector (Invitrogen), and placed under constitutive control of the J23106 promoter (Anderson, C. J. Anderson promoter collection. (2007); Kelly, J. R. et al. Measuring the activity of BioBrick promoters using an in vivo reference standard. J Biol Eng 3, 4, doi: 10.1186/1754-1611-3-4 (2009)), and additionally, a pMW-J23106-nvarkI vector was constructed by adding the His₆ tag and 3×FLAG tag to the C terminal (shown in Tables 2-1 to 2-3). In addition, the T. kodakarensis kptA gene, the E. coli kptA gene, and the S. cerevisiae tpt1 gene were amplified by PCR and cloned into the pQE-80L vector (QIAGEN), the ampicillin resistance cassette (Amp^{r}) of the pQE-80L vector was then substituted with a chloramphenicol resistance cassette (Cam^{r}) to obtain pQE-80LC-tkkptA, pQE-80LC-eckptA, pQE-80LC-sctpt1 (shown in Tables 2-1 to 2-3). The E. coli ΔtrmB/ΔtapT (Kan^{r}) strain was transformed with a pMW-J23106-nvarkI vector, and then transformed with pQE-80LC-tkkptA, pQE-80LC-eckptA, or pQE-80LC-sctpt1. These transformants were inoculated in a 3 mL LB medium to which 20 μg/mL Cam, 50 μg/mL Kan, and 100 μg/mL ampicillin (Amp) were added, and cultured at 37° C. until the logarithmic growth phase was reached. When OD₆₁₀ reached 0.6, in order to induce expression of KptA and Tpt1p, IPTG was added to a final concentration of 10 or 100 UM, and the sample was cultured for 3.5 hours. 1.5 mL of a culture solution was taken, tRNA fractions were extracted, and RNA-MS shotgun analysis was then performed in the same manner as above. The primers, E. coli strains and plasmids used in the experiment are shown in Tables 1 and 2-1 to 2-3. The graphs were created using R (R Foundation).

### 21. Generation of Chemical Structural Formula

The chemical structural formula was generated using ACD/ChemSketch (ACD/Labs) or ChemDraw (PerkinElmer).

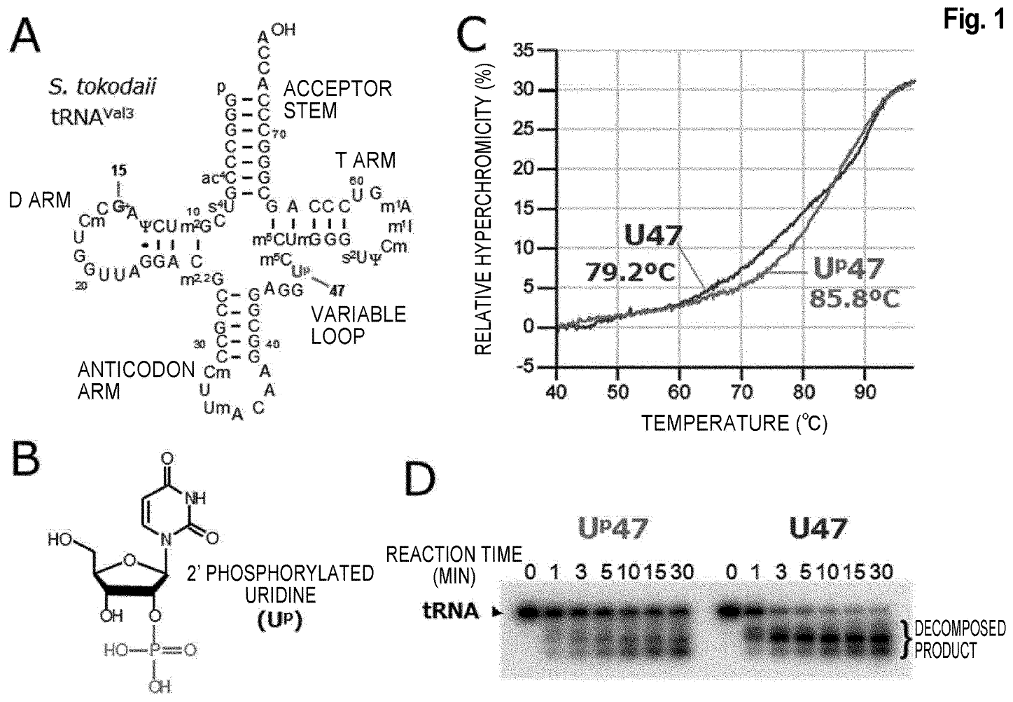

From detailed mass spectrometry (RNA-MS) and biochemical analysis of a plurality of species of tRNAs derived from hyperthermophilic archaea Sulfurisphaera tokodaii isolated and purified as described above, a novel RNA modification, 2′phosphorylated uridine (U^{p}) in which the position 2′ of uridine (U) was phosphorylated at position 47 in the variable loop of tRNA was found (FIGS. 1A and 1B). The U^{p} modification was found in all tRNAs with a variable loop with a length of 5 bases, and introduced at a very high modification rate of 82 to 100%.

In order to examine the effect of the U^{p} modification on the tRNA structure, when the thermal melting temperatures (Tₘ value) of tRNA with the U^{p} modification and tRNA in which the U^{p} modification was enzymatically dephosphorylated were measured, it was found that tRNA with the U^{p} modification had a melting temperature (Tₘ value) that was 6.6° C. higher than tRNA without the U^{p} modification (FIG. 1C). In addition, when comparing the sensitivity to RNase, it was found that the U^{p} modification had a role of protecting tRNA from the degradative enzyme (FIG. 1D).

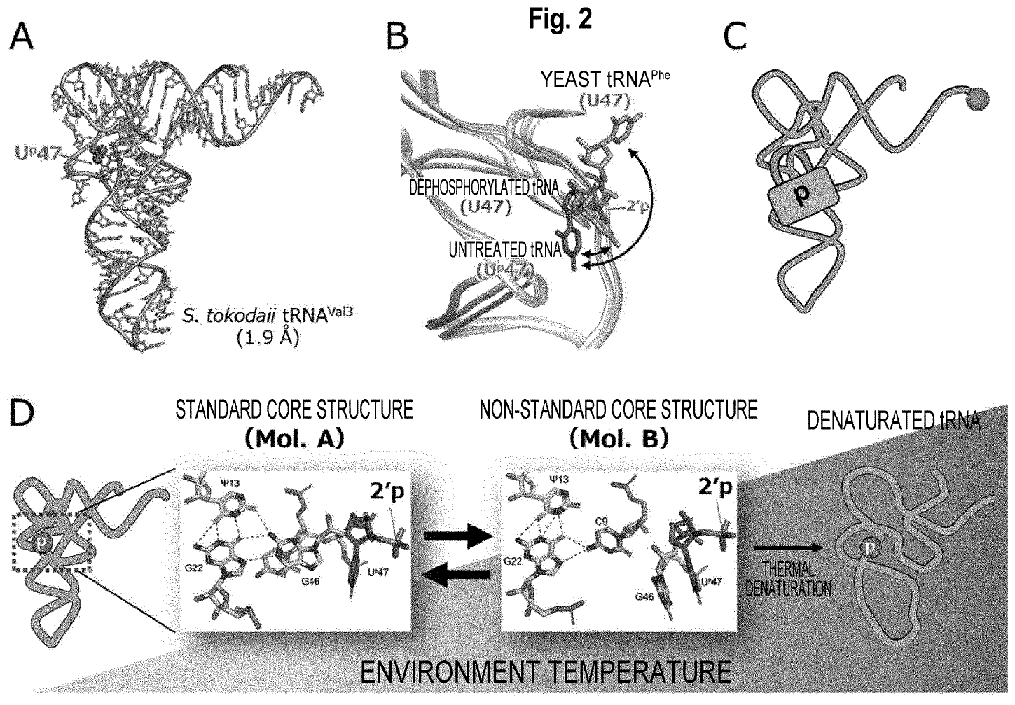

Next, in order to clarify the mechanism of tRNA stabilization by the U^{p} modification, the isolated S. tokodaii tRNA^{Val3} was crystallized, and the X-ray crystal structure was analyzed to obtain a high-resolution (1.9 A) three-dimensional structure (FIG. 2A). Two tRNA molecules with different core structures (Mol. A and Mol. B) were observed in one lattice, and in both structures, the ribose of U^{p} was in C2′-endo conformation. In addition, the 2′ phosphate group protruded toward the solvent, and on the other hand, the uracil base faced the side of the core region of tRNA (FIG. 2B). Since the base moiety at position 47 of tRNA generally faced the side of the solvent, it was suggested that the 2′ phosphate group of U^{p} restricted rotation of the main chain and thus the base moiety was made to face inward (FIG. 2B). Mol. A had a standard core structure of tRNA (FIG. 2D), and a base triple composed of ψ13-G22-G46 was formed, and in Mol. B, G46 was removed from this base triple, and stacked with a uridine base of U^{p}47 (FIG. 2D). In addition, C9 rose up from the lower layer to replace the missing G46, and a new base triple composed of ψ13-G22-C9 was formed (FIG. 2D). It is known that, when tRNA was thermally denatured, it collapsed from the core region (FIG. 2D), and the removal of G46 could be considered as an intermediate state of thermal denaturation. It was though that, when U^{p}47 prevented rotation of the main chain, G46 was accepted, the metastable core structure was stabilized and the thermal denaturation of tRNA was prevented (FIG. 2D). In addition, the crystal structure of tRNA in which U^{p} was dephosphorylated was observed only in Mol. A with the standard core structure, but was not observed in Mol. B. The U^{p} modification did not robustly rigidify and stabilize tRNA, but rather maintained flexibility of the core structure of tRNA, allowed it to have a metastable structure, prevented thermal denaturation of tRNA, and can be said to act like a padlock (FIG. 2C).

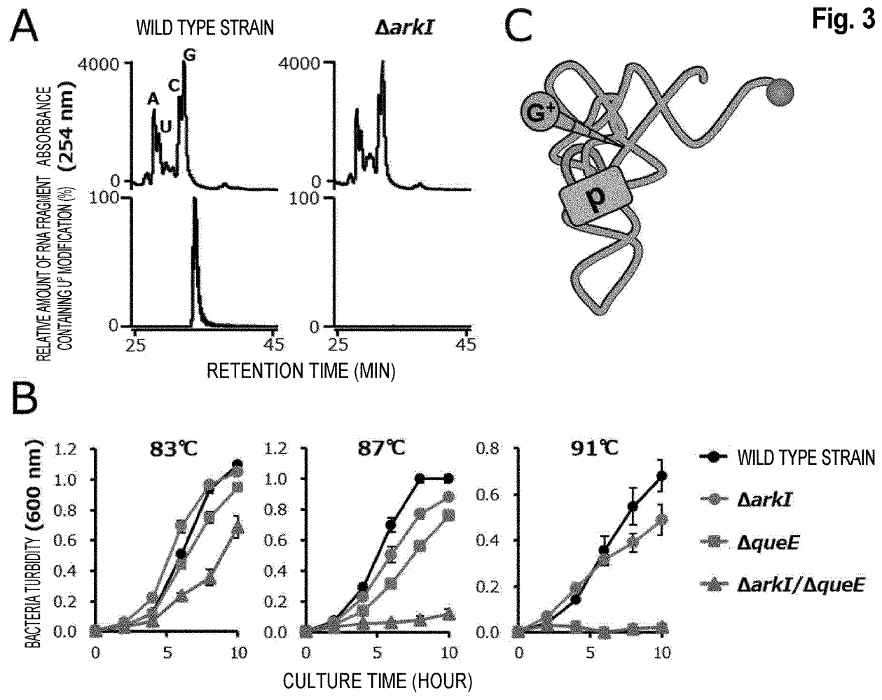

Next, the distribution of species with the U^{p} modification was examined, and based on the results, the U^{p}47 modification enzyme genes were narrowed down by comparative genome analysis. As a result, a protein kinase with unknown functions was narrowed down as a candidate gene. In hyperthermophilic archaea Thermococcus kodakarensis, a knockout strain of this candidate gene was created, LC/MS analysis was performed, and it was found that, in this strain, the U^{p} modification disappeared (FIG. 3A). The study group named this gene arkI (Archaeal RNA kinase). Next, in order to examine the physiological significance of the U^{p} modification, when the growths of the wild type strain and the arkI gene knockout strain (ΔarkI) of T. kodakarensis were compared, ΔarkI exhibited weak high temperature sensitivity (FIG. 3B). In addition, we focused on the archaeosine (G+) modification that imparted rigidity to the tRNA core region (FIG. 1A), created a double knockout strain (ΔarkI/ΔqueE) of the biosynthetic genes queE and arkI and compared their growth (FIG. 3B). As a result, ΔarkI/ΔqueE exhibited a more significant high temperature sensitivity than ΔarkI at 87° C. (FIG. 3B), and it was found that the U^{p} modification in cooperation with the G⁺ modification had an essential role in growth under an extremely high temperature environment. If the U^{p} modification was called a “padlock,” the G⁺ modification acted like a “screw” that rigidly fixed tRNA (FIG. 3C), and it was clearly found in this study that tRNA was stabilized cooperatively by two tRNA modifications with completely different mechanisms of action.

In order to clarify the biochemical properties of ArkI, ArkI (TkArkI) derived from T. kodakarensis was obtained using an E. coli expression system and reconstitution of the U^{p} modification in vitro was performed. As a result, it was found that TkArkI phosphorylated tRNA using ATP as a phosphate group donor (FIG. 4A). It was clearly found from reaction kinetic analysis that TkArkI efficiently recognized and phosphorylated tRNA (Kₘ value 97 nM), but it had a very high Kₘ value of 1.2 mM for ATP and a very low affinity for ATP (FIG. 4B). This result suggested that the rate of the U^{p} modification was limited by the intracellular ATP concentration, and TkArkI sensed the intracellular ATP concentration and phosphorylated tRNA. In addition, TkArkI was crystallized, and the X-ray crystal structure with a resolution of 1.8 A was obtained. TkArkI was composed of two lobes, and similar to the eukaryotic protein kinase (ePK) (FIG. 4C). At the active center of TkArkI, an ATP-binding motif and a motif conserved in ePK involved in phosphate group transfer were observed. It was found through mutant analysis that the residue conserved in these motifs actually contributed to activity (FIG. 4E). In addition, the surrounding of the active center was covered with a positively charged surface (FIG. 4D), and it was found through mutant analysis that these positive charges were necessary for binding to tRNA (FIG. 4E). In addition, guanosine, not ATP, was bound to the active center (FIG. 4C). Since this binding mode differed from an expected ATP binding mode, it was inferred that, when tRNA was bound, a motif that recognized the phosphate group of ATP was positioned at the active center, and ATP could be bound. This finding indicates that TkArkI had a high Kₘ value for ATP.

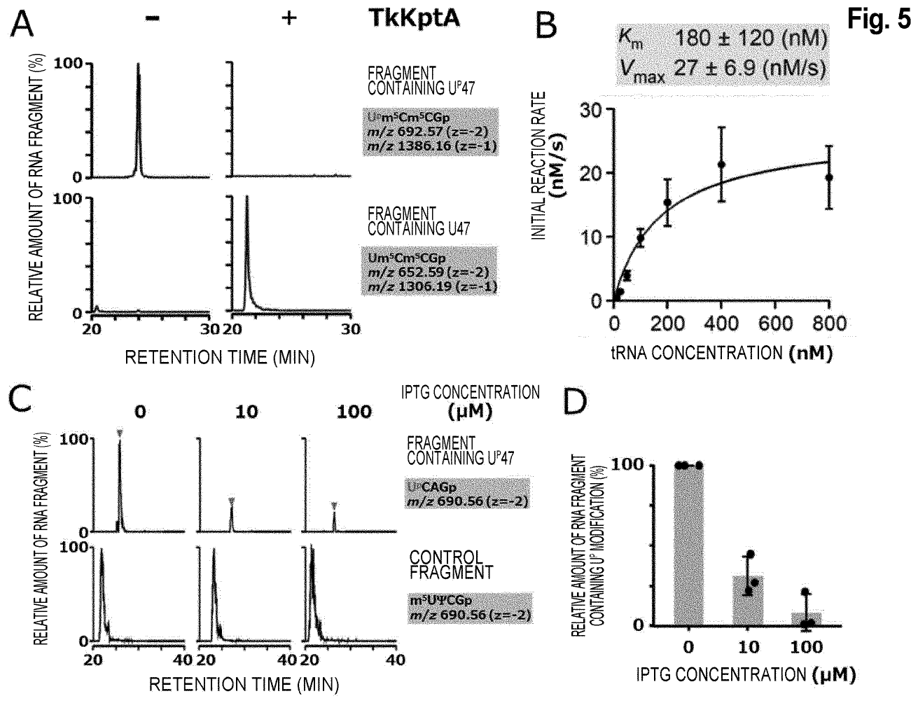

Finally, the reversibility of the U^{p} modification was analyzed. Some organisms with ArkI had the 2′ phosphotransferase KptA, which suggested that the U^{p} modification could be reversible in these organisms. Therefore, KptA (TkKptA) derived from T. kodakarensis was obtained, and biochemical and reaction kinetic analysis was performed. As a result, it was found that TkKptA had a high activity of dephosphorylating the U^{p} modification (FIG. 5A), and the affinity for tRNA was similar to that of TkArkI (FIG. 5B). Next, in order to examine whether KptA actually dephosphorylated the U^{p} modification in cells, when expression of TkKptA was induced while ArkI was expressed in E. coli and the U^{p} modification was introduced into tRNA, it was found that the U^{p} modification was reduced in response to the expression of TkKptA, and KptA actually functioned as an eraser in cells (FIGS. 5C and 5D). This result suggested the presence of an epitranscriptomic gene expression regulation mechanism in which the function of tRNA was regulated by reversible phosphorylation, just as the function of proteins was regulated by reversible phosphorylation. In extremophilic organisms such as hyperthermophilic archaea, the reversibility of the U^{p} modification was thought to enable them to rapidly adapt to sudden changes in the growth temperature by quickly changing flexibility of the tRNA structure.

21. tRNA Phosphorylation Reaction Experiment Using NvArkI with 47-Position Substituted Mutant of tRNA

In order to examine whether ArkI could phosphorylate tRNA mutants in which U at position 47 of tRNA was substituted with C, A, or G, tRNA in which U at position 47 was substituted with C, A, or G was prepared by in vitro transcription synthesis. The template DNA was prepared by PCR using the following primer.

N. viennensis Fw
(SEQ ID NO. 102)
5′-CAGTAATACGACTCACTATAGGGCGGCTGGTCTAGCTCGGTTAT-3′
N. viennensis Body
(SEQ ID NO. 103)
5′-CTGGTCTAGCTCGGTTATGATACCGCTCTTACACAGCGGTGGTC-3′
N. viennensis Rv
(SEQ ID NO. 104)
5′-TGGGCGGCGGGTGATTTGAACACCCGACCACCGCTGTGTAA-3′
N. viennensis Mutant body
(SEQ ID NO. 105)
5′-CTGGTCTAGCTCGGTTATGATACCGCTCTTACACAGCGGTG-3′
N. viennensis Rv_U47C
(SEQ ID NO. 106)
5′-TGGGCGGCGGGTGATTTGAACACCCGGCCACCGCTGTGTAAGAG-3′
N. viennensis Rv_U47A
(SEQ ID NO. 107)
5′-TGGGCGGCGGGTGATTTGAACACCCGTCCACCGCTGTGTAAGAG-3′
N. viennensis Rv_U47G
(SEQ ID NO. 108)
5′-TGGGCGGCGGGTGATTTGAACACCCGCCCACCGCTGTGTAAGAG-3′
S. solfataricus Fw
(SEQ ID NO. 109)
5′-GCTAATACGACTCACTATAAGCCGGGTAGTCTAGTGG-3′
S. solfataricus Body
(SEQ ID NO. 110)
5′-AGCCGGGTAGTCTAGTGGTCAAGGATCCAGGGCTTTGGCCCCTGGG
ACCAGGGTTCGAATCC-3′
S. solfataricus Rv
(SEQ ID NO. 111)
5′-TGGTAGCCGGGCAGGGATTCGAACCCTGGTCC-3′

The U^{p} modification formation reaction was performed in 30 μL of a reaction solution containing 100 mM HEPES-KOH (pH 7.5), 2 mM MgCl₂, 2 mM MnCl₂, 2.5 mM spermidine, 100 mM NaCl, 2 mM DTT, 12U Superase In, 1.1 μM [γ-³²P] ATP (1,500 mCi/mmol; Perkin Elmer), 3 μM NvArkI, and 1.67 UM in vitro transcribed N. viennensis tRNA^{Val} at 45° C. for 3 hours. As a negative control, an in vitro transcription synthesis product of S. solfataricus tRNA^{Gln}, which had no position 47 and a variable loop one base shorter, was used. After the reaction, tRNA was extracted with acidic phenol/chloroform/isoamyl alcohol (25:24:1), collected by precipitation with ethanol, and ATP was then removed using a Centri-Sep spin column. The purified tRNA was isolated by 10% denaturing PAGE, the gel was stained with SYBR Gold for 30 minutes, and the presence of tRNA was confirmed. The gel was exposed to an imaging plate, and ³²P-labeled tRNA was visualized using an FLA-7000 imaging analyzer. The results are shown in FIG. 6.

Based on the results of mutation study, it was found that NvArkI could phosphorylate tRNA even if the base at position 47 was something other than U, and the introduction efficiency was high in the order of U, G, A, and C. On the other hand, tRNA lacking position 47 and with a short V-loop could not be modified. Considering that the U^{p} modification was confirmed only in tRNA with a V-loop of 5 bases in S. tokodaii, it was thought that ArkI recognized and modified the length of the V-loop or the tRNA structure as a substrate rather than the tRNA sequence itself.

In addition, looking at the gene sequences of tRNA derived from various species in the tRNA database, there were a small number of tRNAs in which position 47 was C or A instead of U such as tRNA^{Ile2} derived from N. viennensis and tRNA^{Ala} derived from T. kodakarensis, and a novel modification such as a C^{p} modification and an A^{p} modification could be present. In addition, the presence of an acp³U modification and a D modification at position 47 of tRNA was known, but since these were modifications of the base moiety, ArkI, which was not easily affected by the base, could use these modified bases as substrates and introduce a 2′-phosphate group. However, the substrate specificity of ArkI derived from other organisms was not analyzed, and the length of the V-loop and the stringency for the tRNA structure may differ.

1-25. (canceled)

26. An enzyme comprising an amino acid sequence that (a) is selected from SEQ ID NOs: 1 to 8, or (b) comprises 80% or more sequence identity to a sequence selected from SEQ ID NOs: 1 to 8.

27. A vector comprising a nucleic acid encoding the enzyme according to claim 26.

28. A kit or a composition, which comprises the enzyme according to claim 26 or a nucleic acid that encodes the enzyme.

29. A method for modifying a 2′ hydroxyl group of a nucleotide, which comprises bringing the nucleotide into contact with the enzyme according to claim 26.

30. A method for producing a 2′ phosphorylated nucleoside, which comprises bringing a nucleoside that comprises a 2′ hydroxyl group into contact with the enzyme according to claim 26.

31. A method for stabilizing a polynucleotide, which comprises bringing the polynucleotide into contact with an enzyme having 80% or more sequence identity to an amino acid sequence selected from SEQ ID NOs: 1 to 8.

32. The method according to claim 31, wherein the polynucleotide comprises a nucleotide that comprises a 2′ hydroxyl group.

33. The method according to claim 31, wherein thermostability, resistance to RNase, or both are imparted to the polynucleotide.

34. The method according to claim 31, wherein the polynucleotide is an RNA molecule.

35. The method according to claim 31, wherein the polynucleotide is a tRNA.

36. A method of treating a subject for deficient or reduced mitochondrial tRNA modifications, which comprises stabilizing a polynucleotide in the subject according to the method of claim 31 by administering the enzyme to the subject.
